(19) United States
(12) Patent Application Publication
(10) Pub. No.: US 2024/0395374 A1
(43) Pub. Date: Nov. 28, 2024

(54) PATIENT INFORMATION MANAGEMENT SYSTEMS AND METHODS OF PROCESSING PATIENT INFORMATION BASED ON NON-HEALTHCARE DATA

(71) Applicant: Cerner Innovation, Inc., Kansas City, MO (US)

(72) Inventors: Natalee Agassi, Blue Bell, PA (US); William John Ormerod, JR., Fairfax, VT (US); Changqing Ye, Irvine, CA (US)

(73) Assignee: Cerner Innovation, Inc., Kansas City, MO (US)

(21) Appl. No.: 18/322,296

(22) Filed: May 23, 2023

(51) Int. Cl. G16H 10/60; G16H 50/70

(52) U.S. Cl. CPC G16H 10/60; G16H 50/70

(57) ABSTRACT

In one aspect: A non-healthcare data source associated with a patient may be identified, and a non-healthcare dataset may be extracted from the non-healthcare data source. A target subset of healthcare data may be selected from a set of healthcare data based on the non-healthcare dataset. A query may be executed on the target subset of healthcare data to identify a candidate patient information dataset. A patient information data structure may be created or updated based on the candidate patient information dataset associated with the patient. In another aspect: A match score may be computed between a non-healthcare dataset and a candidate patient information dataset, and responsive to the match score meeting a threshold, the candidate patient information dataset may be selecting as a patient information dataset. The patient information dataset may be stored in the patient information data structure in association with the patient.

### TECHNICAL FIELD

The present disclosure relates to healthcare data, and more particularly to matching healthcare data to patients.

### BACKGROUND

Healthcare data corresponding to a patient may exist at a variety of healthcare data sources. For example, a patient may receive healthcare treatment from different healthcare provider systems, and the respective healthcare provider systems may respectively generate separate healthcare records associated with the patient. Healthcare data from different sources may be matched with a patient; however, erroneous, inaccurate, incomplete, or inconsistently formatted data can make patient-matching difficult. Additionally, a patient's healthcare data associated with different healthcare provider systems may remain unmatched, for example, when one healthcare system is unaware that the patient has additional healthcare data associated with a different system.

The content of this background section should not be construed as prior art merely by virtue of its presence in this section.

### BRIEF DESCRIPTION OF THE DRAWINGS

The embodiments are shown by way of example and not by way of limitation in the figures of the accompanying drawings. It should be noted that references to “an” or “one” embodiment in this disclosure are not necessarily to the same embodiment, and they mean at least one. In the drawings:

FIG. 1 illustrates a system that includes a patient information management system configured to process patient information based on non-healthcare data in accordance with one or more embodiments;

FIG. 2A illustrates example operations that may be performed by the system in accordance with one or more embodiments, including using non-healthcare datasets associated with a patient to determine subsets of healthcare data sources to search for patient information;

FIG. 2B illustrates example operations that may be performed by the system in accordance with one or more embodiments, including using non-healthcare datasets associated with a patient to identify and match patient information datasets with patients;

FIG. 3 illustrates an example embodiment that includes processing patient information data, including identifying and matching patient information data to a patient, based on non-healthcare data; and

FIG. 4 shows a block diagram that illustrates a computer system in accordance with one or more embodiments.

### DETAILED DESCRIPTION

In the following description, for the purposes of explanation, numerous specific details are set forth in order to provide a thorough understanding. Detailed examples are described below for purposes of clarity. One or more embodiments may be practiced without these specific details. Components and/or operations described below should be understood as one specific example which may not be applicable to certain embodiments. Features described in one embodiment may be combined with features described in a different embodiment. In some examples, well-known structures and devices are described with reference to a block diagram form in order to avoid unnecessarily obscuring the present invention. Components and/or operations described below should not be construed as limiting the scope of any of the claims.
1. GENERAL OVERVIEW
2. PATIENT INFORMATION MANAGEMENT SYSTEM
3. OBTAINING PATIENT HEALTHCARE DATA
4. PROCESSING PATIENT INFORMATION
5. PRACTICAL APPLICATIONS, ADVANTAGES & IMPROVEMENTS
6. COMPUTER NETWORKS AND CLOUD NETWORKS
7. MICROSERVICE APPLICATIONS
8. HARDWARE OVERVIEW
9. MISCELLANEOUS; EXTENSIONS

### 1. GENERAL OVERVIEW

The present disclosure provides systems and methods of matching healthcare data to a patient based on non-healthcare data associated with the patient. As used herein, the term “patient” refers to an identified or identifiable person, associated or associable with at least one of non-healthcare data or healthcare data, or with respect to whom non-healthcare data or healthcare data may be collected or utilized.

The non-healthcare data may include digital identity data associated with a patient's digital footprint. The non-healthcare data may include data from various non-healthcare data sources. The non-healthcare data may include data associated with or generated by a patient's social media activity, or data associated with or generated by mobile devices associated with the patient. The non-healthcare data may be utilized to select a target subset of healthcare data from which to search for healthcare data associated with the patient. Digital identity data indicative of a geographic location may be utilized to focus a search for healthcare data that may be associated with a patient to the geographic location indicated by the digital identity data. For example, when a patient travels or relocates to a new location, the patient's healthcare data generated by healthcare provider systems at the new location may be identified based on the non-healthcare data associated with the patient. Additionally, or in the alternative, the non-healthcare data may be utilized to match different healthcare datasets, such as datasets from different healthcare data sources. For example, a first healthcare dataset may have a known association with the patient, and a second healthcare dataset may be associated with the patient based on non-healthcare data associated with the patient. The non-healthcare data may also be used to validate or perform quality assurance with respect to matches between a healthcare dataset and a patient.

This General Overview section is intended to provide a general overview without addressing all aspects of the present disclosure. The full scope of the presently disclosed subject matter is understood from the content of the present disclosure in its entirety.

### 2. PATIENT INFORMATION MANAGEMENT SYSTEM

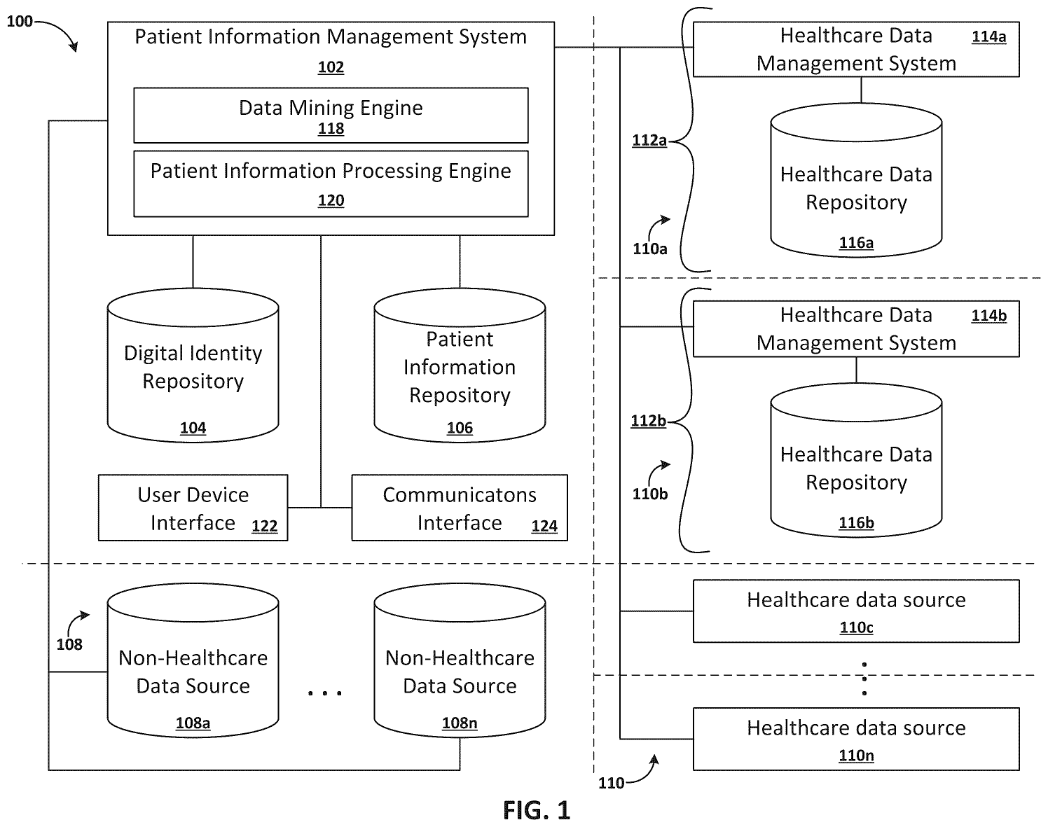

Referring now to FIG. 1, example systems are described. As shown in FIG. 1, a system 100 in accordance with one or more embodiments may be associated with operations in accordance with the present disclosure, and more particularly, operations associated with processing patient information based on non-healthcare data.

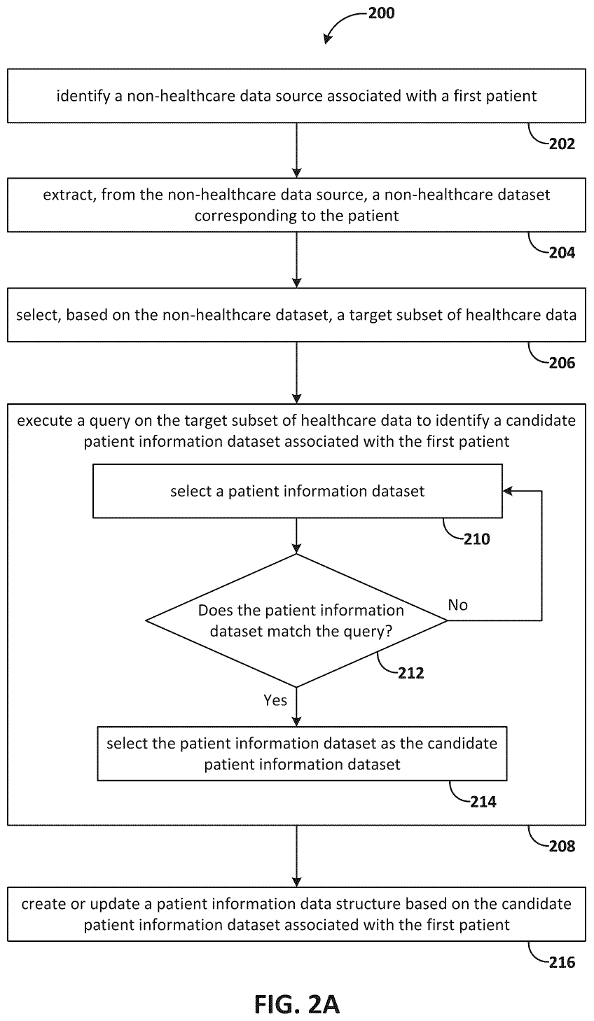

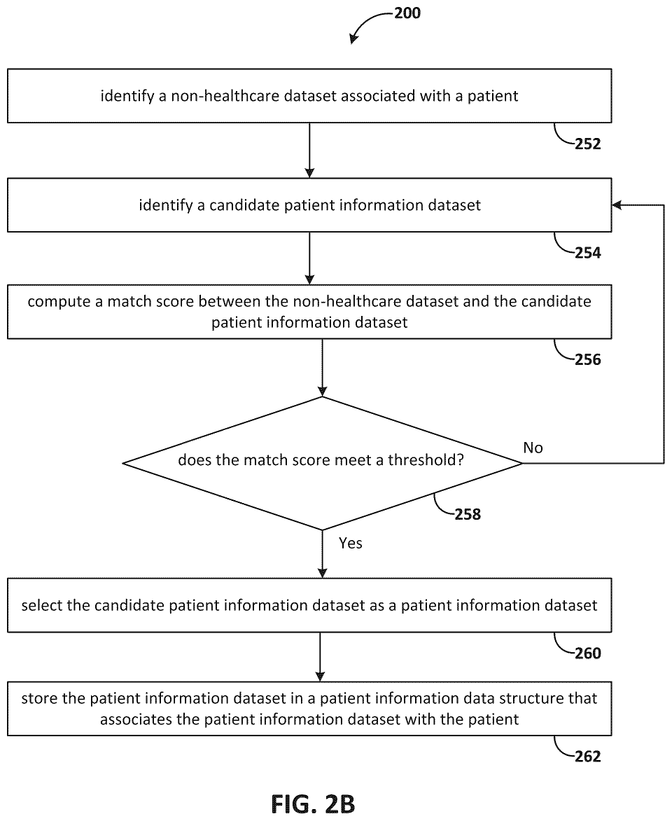

The system 100 may include a patient information management system 102, and at least one of a digital identity repository 104 or a patient information repository 106. The patient information management system 102 may include hardware and/or software configured to carry out the various operations. The various operations may include operations associated with determining, based on non-healthcare data, subsets of healthcare data sources to search for patient information. Additionally, or in the alternative, the various operations may include operations associated with identifying and matching patient information datasets with patients based on non-healthcare data. Examples of operations are further described below, including with reference to FIGS. 2A and 2B.

The patient information management system 102 may be communicatively coupled or couplable with at least one of: the digital identity repository 104 or the patient information repository 106. The digital identity repository 104 may include data, such as non-healthcare data, utilized and/or stored by the patient information management system 102 in association with various operations. The patient information repository 106 may include data, such as healthcare data, utilized and/or stored by the patient information management system 102 in association with various operations.

The digital identity repository 104 and/or the patient information repository 106 may respectively include any type of storage unit and/or device (e.g., a file system, database, collection of tables, or any other storage mechanism) for storing data. Further, the digital identity repository 104 and/or the patient information repository 106 may respectively include multiple different storage units and/or devices. The multiple different storage units and/or devices may or may not be of the same type or located at the same physical site. Further, the digital identity repository 104 and/or the patient information repository 106 may be respectively implemented or executed on the same computing system as the patient information management system 102. Additionally, or in the alternative, the digital identity repository 104 and/or the patient information repository 106 may be respectively implemented or executed on a computing system separate from the patient information management system 102. The digital identity repository 104 and/or the patient information repository 106 may be respectively communicatively coupled to the patient information management system 102 via a direct connection or via a network.

The non-healthcare data stored in the digital identity repository 104 may include a plurality of non-healthcare datasets. The plurality of non-healthcare datasets may be respectively associated with a particular non-healthcare data source from among a plurality of non-healthcare data sources. Additionally, or in the alternative, the plurality of non-healthcare datasets may be respectively associated with a particular patient from among a plurality of patients.

The healthcare data stored in the patient information repository 106 may include a plurality of healthcare datasets. The plurality of healthcare datasets may be respectively associated with a particular healthcare data source from among a plurality of healthcare data sources. Additionally, or in the alternative, the plurality of healthcare datasets may be respectively associated with a particular patient from among a plurality of patients. Additionally, or in the alternative, the patient information data stored in the patient information repository 106 may include a plurality of patient information datasets. The plurality of patient information datasets may be respectively associated with a particular patient from among the plurality of patients.

### A. Non-Healthcare Data and Non-Healthcare Data Sources.

The patient information management system 102 may receive data, such as non-healthcare data, from one or more non-healthcare data sources 108. The non-healthcare data received from a non-healthcare data source 108 may be stored in the digital identity repository 104.

As used herein, the term “non-healthcare data source” refers to a direct or indirect source of non-healthcare data. A non-healthcare data source may include any data source other than a healthcare data source. As examples, non-healthcare data sources may include one or more of: website hosting systems, webservers, social media systems, cloud-computing systems, blockchain systems, telecommunication systems, cellphone tower systems, geolocation systems, mobile device tracking systems, vehicle tracking systems, financial transaction systems, electronic banking systems, utilities systems, travel accommodation systems, airline travel systems, lodging accommodation systems, property rental systems, vehicle rental systems, restaurant management systems, food delivery systems, customs processing systems, security checkpoint systems, public records systems, credit bureau systems, white page systems, or combinations of these.

As used herein, the term “non-healthcare data” refers to data, generated by, stored by, maintained by, or obtained directly or indirectly from, a non-healthcare data source. Non-healthcare data may include additional data generated or derived at least in part from non-healthcare data. Non-healthcare data may include digital identity data. Non-healthcare data does not include healthcare data. The term “non-healthcare dataset” refers to a collection of non-healthcare data. A non-healthcare dataset may include a collection of non-healthcare data associated with a particular non-healthcare data source. A plurality of non-healthcare datasets may each be associated with a different non-healthcare data source from among a plurality of non-healthcare data sources. Additionally, or in the alternative, a non-healthcare dataset may include a collection of non-healthcare data associated with a particular patient. A plurality of non-healthcare datasets may each be associated with a different patient from among a plurality of patients.

As used herein, the term “digital identity data” refers to data associated with a digital identity, digital footprint, or digital presence of a person. The terms “digital identity,” “digital footprint,” and “digital presence” may be used synonymously, and each refers to data generated by, or directly or indirectly associated with, online activity of a person, including data associated with active or passive online activity. In one example, digital identity data generated from a passive online activity may include geolocation data generated by a device that is directly or indirectly associated with a person. In one example, digital identity data generated from an active online activity may include an online post that is directly or indirectly associated with a person. As used herein, the term “digital identity dataset” refers to a collection of digital identity data. A digital identity dataset may be associated with a particular patient. A plurality of digital identity datasets may each be associated with a different patient from among a plurality of patients.

As examples, digital identity data may include data associated with one or more of: website traffic, social medial profiles, social media activity, professional profiles, professional profile activity, job boards, online forums, blogs, online comments, online reviews, IP addresses, cookies, browser history, online search history, photos, videos, cloud-storage data, blockchain data, telecommunications devices, wearable devices, Internet-of-Things devices, cellphone tower transmissions, mobile devices, mobile device tracking data, geolocation devices, vehicle tracking devices, financial transactions, electronic bank transactions, utility bills, utility payments, telecommunications bills, travel accommodations, airline travel, lodging accommodations, property rental, vehicle rentals, restaurants, food deliveries, customs processing data, security checkpoint data, public records, credit bureau information, white page information, or combinations of these. Digital identity data may include personal data associated with the digital presence of a person. Additionally, or in the alternative, digital identity data may include additional data generated or derived at least in part from digital identity data. Additionally, or in the alternative, digital identity data may include personal data associated with, or generated based at least in part on, digital identity data.

A non-healthcare data source 108 may include hardware and/or software configured to carry out the various operations associated with non-healthcare data, including collecting, storing, managing, maintaining, transmitting, and/or receiving non-healthcare data. Additionally, or in the alternative, a non-healthcare data source 108 may include one or more data repositories for storing non-healthcare data. The one or more data repositories may include any type of storage unit and/or device (e.g., a file system, database, collection of tables, or any other storage mechanism) for storing data, including multiple different storage units and/or devices. The multiple different storage units and/or devices may or may not be of the same type or located at the same physical site. A non-healthcare data source 108 may be implemented or executed on one or more computing systems, including a cloud network. The one or more non-healthcare data sources 108 may be communicatively coupled to the patient information management system 102 via a direct connection or via a network.

In one example, the one or more non-healthcare data sources 108 may include a first non-healthcare data source 108a and a second non-healthcare data source 108n. The first non-healthcare data source 108a and the second non-healthcare data source 108n may be separate or unrelated to one another. In one example, the first non-healthcare data source 108a may include actively generated non-healthcare data, such as an online post that is directly or indirectly associated with a patient. In one example, the second non-healthcare data source 108n may include passively generated non-healthcare data, such as geolocation data generated by a device that is directly or indirectly associated with a patient.

The receipt of non-healthcare data, by the patient information management system 102, from the one or more non-healthcare data sources 108, may occur by way of continuous, intermittent, or periodic data transmissions initiated or requested by the patient information management system 102 and/or by the one or more non-healthcare data sources 108. For example, one or more non-healthcare data sources 108 may push non-healthcare data to the patient information management system 102. Additionally, or in the alternative, the patient information management system 102 may query or request non-healthcare data from the non-healthcare data sources 108.

### B. Healthcare Data and Healthcare Data Sources.

The patient information management system 102 may receive healthcare data from one or more healthcare data sources 110. The healthcare data received from a healthcare data source 110 may be stored in the patient information repository 106.

As used herein, the term “healthcare data source” refers to a direct or indirect source of healthcare data. As examples, healthcare data sources may include: healthcare provider systems, health insurance provider systems, health insurance enrollment systems, health insurance organizations, claims and billing systems, patient portal systems, Electronic Health Record (EHR) systems, Health Information Exchange (HIE) systems, Universal Health Record (UHR) systems, personal health record (PHR) systems, Prescription Drug Monitoring Program (PDMP) systems, Regional Health Information Organizations (RHIO) systems, eHealth Exchange systems, public health surveillance organizations, medical research organizations, health tracking devices, health survey systems, government agencies, or mobile-device health systems.

As used herein, the term “healthcare provider system” refers to an identified or identifiable provider of healthcare. As examples, healthcare provider systems may include one or more of: an entity associated with one or more healthcare professionals, a healthcare facility, or a healthcare organization. Additionally, or in the alternative, healthcare provider systems may include one or more of: Care Delivery Organizations (CDOs), hospital networks, medical groups, hospitals, clinics, medical schools, research facilities, or teaching hospitals. Additionally, or in the alternative, healthcare provider system may include entities that provide services in one or more of the following fields: mental health, obstetrics, geriatrics, surgery, disabilities, rehabilitation, optometry, diagnostics, dentistry, podiatry, public health, medical education, or alternative medicine.

As used herein, the term “healthcare professional” refers to a person who provides healthcare treatment or advice based on formal training and experience. As examples, healthcare professionals may include one or more of: physicians, advanced practice providers, physician assistants, allied health professionals, therapists, dictitians, optometrists, pharmacists, pharmacy technicians, medical assistants, physical therapists, occupational therapists, dentists, midwifes, psychologists, public health professionals, or community health professionals. Additionally, or in the alternative, healthcare professionals may include persons who work in one or more of the following fields: mental health, obstetrics, geriatrics, surgery, disabilities, rehabilitation, optometry, diagnostics, dentistry, podiatry, public health, medical education, research, or alternative medicine.

As used herein, the term “healthcare data” refers to patient information data generated by, stored by, maintained by, or obtained directly or indirectly from, a healthcare data source. Healthcare data may include additional data generated or derived at least in part from healthcare data. Healthcare data does not include non-healthcare data. The term “healthcare dataset” refers to a collection of healthcare data. A healthcare dataset may include a collection of healthcare data associated with a particular healthcare data source. A plurality of healthcare datasets may each be associated with a different healthcare data source from among a plurality of healthcare data sources. Additionally, or in the alternative, a healthcare dataset may include a collection of healthcare data associated with a particular patient. A plurality of healthcare datasets may each be associated with a different patient from among a plurality of patients.

As used herein, the term “patient information data” refers to health data, personal data, or a combination of health data and personal data, associated with a particular patient. Patient information data may include healthcare data associated with a particular patient. Patient information data may include health data and/or personal data that is not obtained directly or indirectly from a healthcare data source. For example, patient information data may include health data and/or personal data generated by, stored by, maintained by, or obtained directly or indirectly from, a patient information management system 102. Additionally, or in the alternative, patient information data may include additional patient information data generated or derived at least in part from patient information data. The term “patient information dataset” refers to a collection of patient information data. A patient information dataset may be associated with a particular patient. A plurality of patient information datasets may each be associated with a different patient from among a plurality of patients.

Patient information data may be stored in various formats, including, for example: electronic health records (EHRs), personal health records (PHRs), Universal Health Records (UHRs), or data sets associated with health care data sources. Additionally, or in the alternative, patient information data may be stored in or obtained from data repositories, such as healthcare information repositories, associated with one or more healthcare data sources.

As used herein, the term “health data” refers to data directly or indirectly associated with health or healthcare of a person. As examples, health data may include data associated with one or more of: medical history, diagnoses, conditions, treatments, surgeries, radiology images, test results, medications, allergies, medical device information, mental health conditions, mental health treatments, physical therapy treatments, occupational therapy treatments, genetic information, personal habits, sexual health information, immunizations, health insurance information, vital signs, biometric information, disability status, accommodations, substance use history, reproductive information, physical fitness information, family medical history.

As used herein, the term “personal data” refers to data that relates to an identified or identifiable person. As examples, personal data may include data associated with one or more of: a name of a person, a nickname, a surname, a maiden name, a familial name, a cultural name, a pseudonym, an alias, a user name, a physical address, a mailing address, an email address, a phone number, a social security number, a driver's license number, a passport number, an ID card number, an insurance ID number, a date of birth, place of birth, gender, race, ethnicity, religion, marital status, familial status, spouse or partner name and information, family member name and information, demographic information, educational background, employment history, income information, financial information, citizenship, or immigration status.

The one or more healthcare data sources 110 may be separate or unrelated to one another. For example, the one or more healthcare data sources 110 may be respectively associated with different healthcare provider systems and/or different geographic locations. The one or more healthcare data sources 110 may be communicatively coupled to the patient information management system 102 via a direct connection or via a network. The receipt of healthcare data, by the patient information management system 102, from the one or more healthcare data sources 110, may occur by way of continuous, intermittent, or periodic data transmissions initiated or requested by the patient information management system 102 and/or by the one or more healthcare data sources 110. For example, one or more healthcare data sources 110 may push healthcare data to the patient information management system 102. Additionally, or in the alternative, the patient information management system 102 may query or request healthcare data from the healthcare data sources 110. The healthcare data may be stored in the patient information repository 106.

Additionally, or in the alternative, the patient information management system 102 may transmit data, such as non-healthcare data and/or patient information, to one or more healthcare data sources 110. In one example, non-healthcare data associated with a patient, for example, by the patient information management system 102, may be transmitted to a healthcare data source 110. Additionally, or in the alternative, patient information associated with a patient, for example, based on non-healthcare data, may be transmitted to a healthcare data source 110.

In one example, one or more healthcare data sources 110 may be respectively associated with a healthcare provider system 112, such as an entity associated with one or more healthcare professionals, a healthcare facility, or a healthcare organization. Additionally, or in the alternative, one or more healthcare data sources 110 may be associated with a direct or indirect source of healthcare data other than a healthcare provider system 112. For example, as shown in FIG. 1, the one or more healthcare data sources 110 may include a first healthcare data source 110a associated with a first healthcare provider system 112a, and a second healthcare data source 110b associated with a second healthcare provider system 112b. The first healthcare provider system 112a may include a first healthcare data management system 114a and a first healthcare data repository 116a. The second healthcare provider system 112a may include a second healthcare data management system 114b and a second healthcare data repository 116b. The first healthcare provider system 112a and the second healthcare provider system 112b may be separate or unrelated to one another. Additionally, or in the alternative, the first healthcare data source 110a and the second healthcare data source 110b may be separate or unrelated to one another. For example, the first healthcare provider system 112a and/or the first healthcare data source 110a may correspond to a first geographic location, and the second healthcare provider system 112b and/or the second healthcare data source 110b may correspond to a second geographic location that is different from the first geographic location.

As shown in FIG. 1, a healthcare provider system 112 may include a healthcare data management system 114 and a healthcare data repository 116. The healthcare data management system 114 may include hardware and/or software configured to carry out the various operations pertaining to healthcare data associated with the healthcare provider system 112, including collecting, storing, managing, maintaining, transmitting, and/or receiving healthcare data. The healthcare data associated with the healthcare provider system 112 may include healthcare data associated with patients that have received healthcare treatment from the healthcare provider system 112.

The healthcare data associated with the healthcare provider system 112 may be stored in a healthcare data repository 116. A healthcare data repository 116 may include any type of storage unit and/or device (e.g., a file system, database, collection of tables, or any other storage mechanism) for storing data. Further, a healthcare data repository 116 may include multiple different storage units and/or devices. The multiple different storage units and/or devices may or may not be of the same type or located at the same physical site. Further, the healthcare data repository 116 may be implemented or executed on the same computing system as the healthcare data management system 114. Additionally, or in the alternative, the healthcare data repository 116 may be implemented or executed on a computing system separate from the healthcare data management system 114. The healthcare data repository 116 may be communicatively coupled to the healthcare data management system 114 via a direct connection or via a network. Additionally, or in the alternative, the healthcare data management system 114 may be communicatively coupled to the one or more patient information management system 102 via a direct connection or via a network.

In one example, the one or more healthcare data sources 110 may include one or more source other than a healthcare provider system 112. For example, a third healthcare data source 110c and/or a fourth healthcare data source 110n may represent a source of healthcare data other than a healthcare provider system 112. The third healthcare data source 110c and the fourth healthcare data source 110n may be separate or unrelated to one another. Additionally, or in the alternative, the third healthcare data source 110c and/or the fourth healthcare data source 110n may be separate or unrelated to at least one of: the first healthcare data source 110a or the second healthcare data source 110b. For example, the third healthcare data source 110c may correspond to a third geographic location, and the fourth healthcare data source 110n may correspond to a fourth geographic location that is different from the third geographic location. The third geographic location and/or the fourth geographic location may be different from at least one of: the first geographic location or the second geographic location.

In one example, the third healthcare data source 110c and the fourth healthcare data source 110n may correspond to a different kind of healthcare data from one another. Additionally, or in the alternative, at least one of the third healthcare data source 110c or the fourth healthcare data source 110n may correspond to a different kind of healthcare data from the first healthcare data source 110a or the second healthcare data source 110b. In one example, the third healthcare data source 110c may include at least one of: a health insurance provider system, a health insurance enrollment system, a health insurance organization, a claims and billing system, a patient portal system, an EHR system, an HIE system, a UHR system, a PHR system, a PDMP system, a RHIO system, or an eHealth Exchange system. In one example, the fourth healthcare data source 110n may include at least one of: a public health surveillance organization, a medical research organization, a health tracking device, a health survey system, a government agency, or a mobile-device health system.

As used herein, the term “geographic location” may include one or more of: a city, a town, a state, a county, a country, a region, a building, a transportation hub, a place of business, a road, a park, a landmark, a natural physical feature (e.g., a mountain, a lake, etc.). Additionally, or in the alternative, the term “geographic location” may include geolocation coordinates, such as a latitude and a longitude. Additionally, or in the alternative, the term geographic location may include an address, a cross-street, or an area defined by a perimeter or a radius.

### C. Data Mining Engine.

Referring further to FIG. 1, the patient information management system 102 may include a data mining engine 118. The data mining engine 118 may obtain non-healthcare data from one or more non-healthcare data sources 108. The non-healthcare data obtained by the data mining engine 118 may be associated with one or more identified or identifiable patients. A patient who is to be associated with non-healthcare data may be identified prior to the data mining engine 118 obtaining the non-healthcare data. Additionally, or in the alternative, non-healthcare data may be subsequently associated with a patient after the non-healthcare data is obtained by the data mining engine 118. The data mining engine 118 may store non-healthcare data in a digital identity data structure that associates the non-healthcare data with at least one of: a patient, or a non-healthcare data source 108.

In one example, the data mining engine 118 may obtain non-healthcare data based on information that identifies one or more non-healthcare data sources 108 from which non-healthcare data associated with a patient may be obtained. The data mining engine 118 may obtain non-healthcare data from the one or more non-healthcare data sources 108 identified based on the information and may store the non-healthcare data in the digital identity repository 104, for example, in a digital identity data structure that associates the non-healthcare data with the patient.

In one example, the information that identifies the one or more non-healthcare data sources 108 may be obtained from a healthcare provider system 112. Additionally, or in the alternative, the information may be obtained from a user device interface, for example, responsive to a user input. For example, a patient may authorize the collection of non-healthcare data pertaining to the patient. The authorization may identify one or more non-healthcare data sources 108 from which the non-healthcare data may be obtained. In one example, a patient may provide access credentials associated with one or more non-healthcare data sources 108, such as a username and/or a password associated with a respective non-healthcare data source 108.

The data mining engine 118 may proceed, for example, responsive to the patient's authorization, to automatically obtain non-healthcare data from the one or more non-healthcare data sources 108. Additionally, or in the alternative, responsive to the patient's authorization, the healthcare provider system 112 may automatically provide information that identifies the one or more non-healthcare data sources 108 to the patient information management system 102. Additionally, or in the alternative, the information that identifies the one or more non-healthcare data sources 108 may be provided to the patient information management system 102 via the user device interface. The data mining engine 118 may proceed to obtain non-healthcare data from the one or more non-healthcare data sources 108 identified by the information from the healthcare provider system 112 and/or from the input via the user device interface.

In one example, the data mining engine 118 may search one or more non-healthcare data sources 108 for non-healthcare data associated with a patient. For example, the patient information management system 102 may not possess information that identifies specific non-healthcare data sources 108 from which non-healthcare data associated with the patient may be obtained. The data mining engine 118 may execute queries to search one or more non-healthcare data sources 108 based on one or more items of personal data associated with a patient. Additionally, or in the alternative, the data mining engine 118 may supplement or augment existing non-healthcare data associated with the patient obtained from a first one or more non-healthcare data sources 108, by searching one or more second non-healthcare data sources 108 for additional non-healthcare data associated with the patient. Non-healthcare data associated with a patient obtained by the data mining engine 118, for example, as a result of one or more queries, may be stored in the digital identity repository 104, for example, in a digital identity data structure that associates the non-healthcare data with the patient.

In one example, the data mining engine 118 may obtain non-healthcare data, from one or more non-healthcare data sources 108, that are unassociated with a particular patient. The data mining engine 118 may store non-healthcare data this is unassociated with a particular in the digital identity repository 104, for example, in a digital identity data structure that associates the non-healthcare data with an identifier representing an unidentified patient. One or more patients may be subsequently associated with portions of the non-healthcare data stored in the digital identity repository 104. For example, the data mining engine 118 may execute queries to search the digital identity repository 104 based on one or more items of personal data associated with a patient. Non-healthcare data located by the data mining engine 118, for example, as a result of one or more queries corresponding to a patient, may be associated with the patient in the digital identity data structure.

### D. Patient Information Processing Engine.

Referring further to FIG. 1, the patient information management system 102 may include a patient information processing engine 120. The patient information processing engine 120 may obtain healthcare data from one or more healthcare data sources 110. The healthcare data obtained by the patient information processing engine 120 may be associated with one or more identified or identifiable patients. A patient who is to be associated with healthcare data may be identified prior to the patient information processing engine 120 obtaining the healthcare data. Additionally, or in the alternative, healthcare data may be subsequently associated with a patient after the healthcare data is obtained by the patient information processing engine 120. The patient information processing engine 120 may store healthcare data in a patient information data structure that associates the healthcare data with at least one of: a patient, or a healthcare data source 110.

In one example, the patient information processing engine 120 may obtain healthcare data based on information that identifies one or more healthcare data sources 110 from which healthcare data associated with a patient may be obtained. In one example, the patient information processing engine 120 may identify one or more healthcare data sources 110 from which healthcare data associated with a patient may be obtained based at least in part on non-healthcare data associated with the patient. For example, based on the non-healthcare data, the patient information processing engine 120 may identify a geographic location associated the patient, and based on the geographic location, the patient information processing engine 120 may identify a subset of healthcare data sources 110 to search for healthcare data. In one example, the geographic location may be a location that is newly associated with the patient, such as a geographic location where the patient may be vacationing or traveling, or to which the patient may have relocated. Additionally, or in the alternative, the geographic location may be different from a known geographic location associated with the patient, such as the patient's last known address. The patient information processing engine 120 may obtain healthcare data from the one or more healthcare data sources 110 identified based on the information, such as the non-healthcare data, and may store the healthcare data in the patient information repository 106, for example, in a patient information data structure that associates the healthcare data with the patient.

The patient information processing engine 120 may automatically obtain healthcare data from one or more healthcare data sources 110 associated with a patient, for example, responsive to having identified the one or more healthcare data sources 110 based at least in part on non-healthcare data associated with the patient. Additionally, or in the alternative, the patient information processing engine 120 may search one or more healthcare data sources 110 for healthcare data associated with a patient. For example, the patient information management system 102 may not possess information that identifies specific healthcare data sources 110 from which healthcare data associated with the patient may be obtained. The patient information processing engine 120 may execute queries to search one or more healthcare data sources 110 based on non-healthcare data, such as digital identity data, associated with the patient. Additionally, or in the alternative, the patient information processing engine 120 may determine a subset of healthcare data sources 110 to search based on non-healthcare data, such as digital identity data, associated with the patient.

In one example, the patient information processing engine 120 may match healthcare data to a patient based on non-healthcare data associated with the patient. Additionally, or in the alternative, the patient information processing engine 120 may supplement or augment existing healthcare data associated with the patient obtained from a first one or more healthcare data sources 110, by searching one or more second healthcare data sources 110 for additional healthcare data associated with the patient. Additionally, or in the alternative, the patient information processing engine 120 may generate or populate healthcare data associated with a patient based on non-healthcare data associated with the patient.

Healthcare data associated with a patient obtained or generated by the patient information processing engine 120, for example, as a result of one or more queries, may be stored in the patient information repository 106, for example, in a patient information data structure that associates the healthcare data with the patient.

In one example, the patient information processing engine 120 may obtain healthcare data, from one or more healthcare data sources 110, that are unassociated with a particular patient. The patient information processing engine 120 may store healthcare data this is unassociated with a particular in the patient information repository 106, for example, in a patient information data structure that associates the healthcare data with an identifier representing an unidentified patient. One or more patients may be subsequently associated with portions of the healthcare data stored in the patient information repository 106. For example, the patient information processing engine 120 may execute queries to search the patient information repository 106 based on non-healthcare data, such as digital identity data, associated with a patient. Healthcare data located by the patient information processing engine 120, for example, as a result of one or more queries corresponding to a patient, may be associated with the patient in the patient information data structure.

In one example, the patient information processing engine 120 may match healthcare data with a patient, or with other healthcare data associated with the patient, based on non-healthcare data associated with the patient. The match may be determined based at least in part on digital identity data associated with the patient obtained from one or more non-healthcare data sources 108. The patient information processing engine 120 may determine a match score between healthcare data, such as a patient information dataset associated with an unknown patient, and a known patient information dataset associated with a known patient. The healthcare data, such as the patient information dataset associated with the unknown patient, may be obtained from one or more healthcare data sources 110. Healthcare data, such as the patient information dataset associated with the unknown patient, that meets a threshold match score may be associated with the patient, for example, in the patient information data structure. The known patient information dataset may be based at least in part on non-healthcare data associated with the patient and/or patient information obtained from a different healthcare data source 110. In one example, the healthcare data may include a plurality of patient information datasets from which candidate patient information datasets may be selected and compared to the known patient information dataset to determine a match score between them. A candidate patient information dataset that has a match score that meets the threshold may be associated with the patient.

In one example, a non-healthcare dataset utilized by the patient information processing engine 120 to search for healthcare data may be validated by comparing healthcare data obtained based on the non-healthcare dataset to a second non-healthcare dataset. The patient information processing engine 120 may determine a match score between a patient information dataset, obtained based on a first non-healthcare dataset, and a second non-healthcare dataset. The patient information dataset may be associated with the patient, and, for example, stored in the patient information data structure, based on the match score with the second non-healthcare dataset meeting a threshold match score.

In one example, patient information datasets that have been matched with respective patients may be validated based on non-healthcare data. For example, the patient information processing engine 120 may generate a confidence metric that indicates a confidence level of a match between a patient and healthcare data, such as a patient information dataset. The confidence metric may be determined by computing a match score between a non-healthcare dataset associated with a patient, such as a digital identity dataset associated with the patient, and a candidate patient information dataset that has been associated with the patient. The confidence metric may be generated based on the match score and may be stored in the patient information data structure in association with the candidate patient information dataset.

### E. Matching Algorithms.

The patient information management system 102 may utilize various suitable models or algorithms to search for non-healthcare data and/or healthcare data, and/or to match non-healthcare data and/or healthcare data with patients. The models or algorithms may include one or more machine learning models or algorithms, and/or one or more classical models or algorithms. A machine learning model may include one or more machine-learning algorithms configured to automatically learn relevant patterns and relationships in data, for example, without the need for manual feature selection or strong assumptions about the data distribution. A classical model may include one or more classical statistical algorithms that rely on a set of assumptions about one or more of the underlying data, the data generating process, or the relationships between the variables. Example classical statistical algorithms may include linear regression, logistic regression, ANOVA (analysis of variance), or hypothesis testing.

In one or more embodiments, a machine learning model may include one or more machine learning algorithms that can be iterated to learn a target model f that best maps a set of input variables to an output variable. In particular, a machine learning algorithms may be configured to generate and/or train a machine learning model.

A machine learning algorithms can be iterated to learn a target model f that best maps a set of input variables to an output variable, using a set of training data. The training data may include datasets and associated labels. The datasets may be associated with input variables for the target model f. The associated labels may be associated with the output variable of the target model f. The training data may be updated based on, for example, feedback on the accuracy of the current target model f. Updated training data may be fed back into the machine learning algorithms, which in turn updates the target model f.

A machine learning algorithm may generate a target model/such that the target model f best fits the datasets of training data to the labels of the training data. Additionally, or alternatively, a machine learning algorithm may generate a target model f such that when the target model f is applied to the datasets of the training data, a maximum number of results determined by the target model/matches the labels of the training data. Different target models may be generated based on different machine learning algorithms and/or different sets of training data.

A machine learning algorithm may include supervised algorithms and/or unsupervised algorithms. Various types of algorithms may be used, such as linear regression, logistic regression, linear discriminant analysis, classification and regression trees, naïve Bayes, k-nearest neighbors, learning vector quantization, support vector machine, bagging, and random forest, boosting, backpropagation, and/or clustering.

In one or more embodiments, the system 100 may include a model trainer that includes one or more machine learning algorithms configured to generate and/or train a machine learning model. In one example, the model trainer may obtain a plurality of training data sets, such as from the digital identity repository 104 and/or the patient information repository 106. The model trainer may train a machine learning model utilized by the patient information management system 102 based at least in part on the plurality of training data sets. In one example, the model trainer may train a machine learning model utilized by the data mining engine 118. In one example, the model trainer may train a machine learning model utilized by the patient information processing engine 120.

The plurality of training data sets may respectively include one or more patient information datasets associated with a patient that were obtained based on non-healthcare data, such as a digital identity dataset associated with the patient. Additionally, or in the alternative, the plurality of training data sets may respectively include one or more matches between a patient information dataset and a patient made based on non-healthcare data, such as a digital identity dataset. Additionally, or in the alternative, the plurality of training data sets may respectively include one or more matches between a first patient information dataset associated with an unknown patient, obtained based on non-healthcare data, and a second patient information dataset having a known association and a patient, made based on non-healthcare data, such as a digital identity dataset.

The one or models utilized by the patient information management system 102 may include deterministic models, probabilistic models, fuzzy models, or hybrid models that include a combination of models. In one example, the patient information management system 102 may utilize one or more of: a Fellegi-Sunter mode, a Jaro-Winkler Distance model, a Levenshtein Distance model, a Soundex model, a Double Metaphone model, a Bayesian Networks model, or a Support Vector Machines (SVM) model.

### F. System Interfaces.

Referring further to FIG. 1, the system 100 may include a user device interface 122 communicatively coupled or couplable with at least one of: the patient information management system 102, the digital identity repository 104, or the patient information repository 106. Additionally, or in the alternative, the user device interface 122 may be communicatively coupled or couplable with one or more healthcare data sources 110, and/or with one or more non-healthcare data sources 108. The user device interface 122 may include hardware and/or software configured to facilitate interactions between a user and various aspects of the system 100. The user device interface 122 may render user interface elements and receive input via user interface elements. Examples of interfaces include a graphical user interface (GUI), a command line interface (CLI), a haptic interface, and a voice command interface. Examples of user interface elements include checkboxes, radio buttons, dropdown lists, list boxes, buttons, toggles, text fields, date and time selectors, command lines, sliders, pages, and forms.

In an embodiment, different components of a user device interface 122 may be specified in different languages. The behavior of user interface elements may be specified in a dynamic programming language, such as JavaScript. The content of user interface elements may be specified in a markup language, such as hypertext markup language (HTML) or XML User Interface Language (XUL). The layout of user interface elements may be specified in a style sheet language, such as Cascading Style Sheets (CSS). Alternatively, the user device interface 122 may be specified in one or more other languages, such as Java, C, or C++.

As further shown in FIG. 1, the system 100 may include a communications interface 124 communicatively coupled or couplable with at least one of: the patient information management system 102, the digital identity repository 104, or the patient information repository 106. Additionally, or in the alternative, the communications interface 124 may be communicatively coupled or couplable with one or more healthcare data sources 110, and/or with one or more non-healthcare data sources 108. The communications interface 124 may include hardware and/or software configured to transmit data to and/or from the system 100, and or between respective components of the system 100. For example, the communications interface 124 may transmit and/or receive data between and/or among the patient information management system 102, the digital identity repository 104, the patient information repository 106, the one or more non-healthcare data sources 108, the one or more healthcare data sources 110, or the user device interface 122. Additionally, or in the alternative, the communications interface 124 may be communicatively coupled or couplable with one or more external resources that utilize, or that are utilized by, the patient information management system 102.

In one example, the patient information management system 102 may be implemented on one or more digital devices. The term “digital device” generally refers to any hardware device that includes a processor. A digital device may refer to a physical device executing an application or a virtual machine. Examples of digital devices include a computer, a tablet, a laptop, a desktop, a netbook, a server, a web server, a network policy server, a proxy server, a generic machine, a function-specific hardware device, a hardware router, a hardware switch, a hardware firewall, a hardware firewall, a hardware network address translator (NAT), a hardware load balancer, a mainframe, a television, a content receiver, a set-top box, a printer, a mobile handset, a smartphone, a personal digital assistant (PDA), a wireless receiver and/or transmitter, a base station, a communication management device, a router, a switch, a controller, an access point, and/or a client device.

In one or more embodiments, the system 100 may include more or fewer components than the components shown in FIG. 1. The components shown in FIG. 1 may be local to or remote from each other. The components shown in FIG. 1 may be implemented in software and/or hardware. Each component may be distributed over multiple applications and/or machines. Multiple components may be combined into one application and/or machine. Operations described with respect to one component may instead be performed by another component.

### 3. OBTAINING PATIENT HEALTHCARE DATA

Referring now to FIGS. 2A and 2B, example operations 200 associated with processing patient information data in accordance with one or more embodiments are further described. The operations 200 described with reference to FIGS. 2A and 2B may be associated with one or more components of the patient information management system 102. One or more operations shown in FIGS. 2A and/or 2B may be modified, rearranged, or omitted all together. Accordingly, the particular sequence of operations shown in FIGS. 2A and 2B should not be construed as limiting the scope of one or more embodiments.

### A. Determining Subsets of Healthcare Data Sources to Search for Patient Information.

As shown in FIG. 2A, example operations 200 may be performed to determine subsets of healthcare data sources to search for patient information. The operations 200 may include using non-healthcare datasets associated with a patient to determine subsets of healthcare data sources to search for patient information associated with the patient. As shown in FIG. 2A, the operations 200 may include, at block 202, identifying a non-healthcare data source associated with a first patient. The non-healthcare data source may be identified by the data mining engine 118 described with reference to FIG. 1. For example, the data mining engine 118 may identify the non-healthcare data source associated with the first patient from a data structure in a digital identity repository that associates the non-healthcare data source with the first patient.

At block 204, the operations 200 may include extracting, from the non-healthcare data source, a non-healthcare dataset corresponding to the first patient. The non-healthcare dataset may include location data associated with the first patient. The location data may indicate a geographic location where the first patient may have received healthcare treatment. The geographic location may be allocation where the patient may be vacationing or traveling, or to which the patient may have relocated. At block 204, the operations 200 may include extracting non-healthcare datasets corresponding to the first patient from multiple non-healthcare data sources. The non-healthcare datasets may be combined, consolidated, filtered, or the like.

At block 206 the operations 200 may include selecting, based on the non-healthcare dataset, a target subset of healthcare data. The target subset of healthcare data may include healthcare data associated with one or more healthcare data sources corresponding to the geographic location indicated by the location data associated with the first patient.

In one example, the target subset of healthcare data may be selected from a set of healthcare data based on digital identity data in the non-healthcare dataset corresponding to the first patient. For example, the operations 200 at block 206 may include extracting digital identity data, corresponding to the first patient, from the non-healthcare dataset corresponding to the patient, and selecting the target subset of healthcare data based on the digital identity data corresponding to the first patient.

In one example, a plurality of target subsets of healthcare data selected at block 206 based on the non-healthcare dataset. The plurality of target subsets of healthcare data may respectively correspond to the geographic location indicated by the non-healthcare dataset corresponding to the first patient. For example, each of the plurality of target subsets of healthcare data may correspond to a different healthcare data source associated with the geographic location indicated by the non-healthcare dataset corresponding to the first patient.

At block 208 the operations 200 may include executing a query on the target subset of healthcare data to identify a candidate patient information dataset associated with the first patient. The query used to identify the candidate patient information dataset may be based on digital identity data included in the non-healthcare dataset associated with the first patient, such as personal data in the digital identity data. The query may identify the candidate patient information dataset based on personal data in the candidate patient information dataset that matches the query.

In one example, the candidate patient information dataset may be identified based at least in part on a comparison of the digital identity data of the non-healthcare dataset with the personal data of the candidate subset of healthcare data. For example, as shown in FIG. 2A, at block 210, the operations 200 may include selecting a patient information dataset from the target subset of healthcare data. At block 212, the operations 200 may include determining whether the patient information dataset matches the query. If the patient information dataset does not match the query, the operations 200 may include refraining from selecting the patient information dataset, and the operations 200 may return to block 210, to select a next patient information dataset. If the patient information dataset matches the query, then at block 214, the operations may include selecting the patient information dataset as the candidate patient information dataset. In one example, a plurality of candidate patient information datasets associated with the first patient may be identified at block 208. The plurality of candidate patient information datasets may respectively correspond to one or more healthcare data sources. At block 216, the operations 200 may include creating or updating a patient information data structure, such as in a patient information repository, based on the candidate patient information dataset associated with the first patient.

In one example, subsequent to identifying the candidate patient information dataset, the operations 200 may include computing a match score between (a) known patient information dataset associated with the first patient and (b) the candidate patient information dataset. The operations 200 may further include determining that the match score meets a threshold, and the patient information data structure may be created or updated based on the candidate patient information dataset responsive to determining that the match score meets the threshold. Additionally, or in the alternative, if the match score does not meet the threshold, the operations 200 may include determining that the candidate patient information dataset does not correspond to the first patient. The operations 200 may include refraining from creating or updating the patient information data structure based on the candidate patient information dataset if the match score for the candidate patient information dataset does not meet the threshold. Additionally, or in the alternative, if the match score for the candidate patient information dataset does not meet the threshold, the operations 200 may include identifying a next candidate patient information dataset at block 208. Additionally, or in the alternative, a patient information data structure may be created or updated, responsive to the candidate patient information dataset not meeting the threshold, to include an indication that the candidate patient information dataset is unassociated with the patient.

In one example, the operations 200 may include augmenting a patient information dataset based on non-healthcare data, such as digital identity data, to generate an augmented patient information dataset. The augmented patient information dataset may be stored in the patient information data structure in association with the first patient. Additionally, or in the alternative, the operations 200 may include augmenting an additional patient information dataset based on the augmented patient information dataset. The additional augmented patient information dataset may be stored in the patient information data structure in association with the first patient.

B. Identifying and Matching Patient Information Datasets with Patients.

As shown in FIG. 2B, example operations 200 may be performed to identify and match patient information datasets with patients based on non-healthcare datasets associated with respective patients. Example operations 200 may include, at block 252, identifying a non-healthcare dataset associated with a patient, and at block 254, identifying a candidate patient information dataset. The non-healthcare dataset may be identified in a digital identity repository or from a non-healthcare data source. The candidate patient information dataset may be identified in a patient information repository or from a healthcare data source.

At block 256, the operations 200 may include computing a match score between the non-healthcare dataset and the candidate patient information dataset. At block 258, the operations 200 may include determining whether the match score meets a threshold. If the threshold is not met, the operations 200 may return to block 254, where a next candidate patient information dataset may be selected. If the threshold is met, the operations 200 may proceed to block 260, by selecting, responsive to the match score meeting the threshold, the candidate patient information dataset as a patient information dataset. At block 262, the operations 200 may include storing the patient information dataset in a patient information data structure that associates the patient information dataset with the patient.

In one example, the match score between the non-healthcare dataset and the first candidate patient information dataset may be computed based on digital identity data in the non-healthcare dataset corresponding to the patient. For example, the operations 200 at block 256 may include extracting digital identity data, corresponding to the patient, from the non-healthcare dataset corresponding to the patient, and computing the match score between the digital identity data and the candidate patient information dataset.

In one example, the operations 200 may include transmitting data to at least one healthcare provider system. For example, responsive to a match score meeting the threshold at block 258, the operations 200 may include transmitting to at least one healthcare provider system, at least one of: an indication that the match score meet the threshold, an indication that the patient information dataset that met the threshold has been associated with the first patient, or an augmented patient information dataset, associated with the first patient, generated based at least in part on digital identity data extracted from the non-healthcare dataset. Additionally, or in the alternative, responsive to a match score falling below the threshold at block 258, the operations 200 may include transmitting to at least one healthcare provider system, at least one of: an indication that the second match score falls below the threshold, an indication that the patient information dataset is unassociated with the patient, or an augmented patient information dataset unassociated with the patient, such as an augmented patient information dataset generated to remove an association with the patient.

### C. Using Non-Healthcare Data to Validate Patient Information Identified by the System.

In one example, operations 200 may include populating a patient information dataset based on healthcare data from one or more healthcare data sources located based on a non-healthcare dataset. Prior to populating the patient information dataset, the healthcare data from one or more healthcare data sources may be verified based on an additional non-healthcare dataset.

In one example, the operations 200 may include identifying a first non-healthcare dataset associated with a first patient and extracting a first set of digital identity data from the first non-healthcare dataset. The operations 200 may include executing a first query on at least one first healthcare data source based on the first set of digital identity data and receiving a first query result identifying a first candidate patient information dataset associated with a first patient. The operations 200 may include computing a first match score between a second non-healthcare dataset associated with the first patient and the first candidate patient information dataset. Responsive to the first match score meeting a threshold, the operations may include creating or updating a first patient information data structure, associated with the first patient, based on the first candidate patient information dataset.

Additionally, or in the alternative, the operations may include identifying a second non-healthcare dataset associated with a second patient and extracting a second set of digital identity data from the second non-healthcare dataset. The operation 200 may include executing a second query on at least one second healthcare data source based on the second set of digital identity data and receiving a second query result identifying a second candidate patient information dataset associated with a second patient. The operations 200 may include computing a second match score between a third non-healthcare dataset associated with the second patient and the second candidate patient information dataset. Responsive to the second match score falling below the threshold, the operations 200 may include refraining from creating or updating a second patient information data structure, associated with the second patient, based on the second candidate patient information dataset.

### D. Using Non-Healthcare Data for Quality Assurance.

In one example, operations 200 may include checking match results based on a non-healthcare dataset. For example, the operations 200 may include identifying healthcare datasets that may have been erroneously matched with a patient. Additionally, or in the alternative, the operations 200 may include identifying healthcare datasets that may be associated with a patient but that may have been erroneously identified as non-matching to the patient. Additionally, or in the alternative, the operations 200 may include confirming that healthcare datasets that have been matched with a patient are correct matches, or that healthcare datasets that have been identified as non-matching to patients are correct non-matches.

With respect to identifying healthcare datasets that may have been erroneously matched with a patient, the operations 200 may include identifying a patient information dataset that has been matched to a patient, and a non-healthcare dataset associated with the patient. The operations 200 may include computing a match score between the matched patient information dataset and the non-healthcare dataset, such as digital identity data extracted from the non-healthcare dataset. Responsive to the match score falling below a threshold, the operations 200 may include generating a confidence metric indicating a confidence level of the patient information data subset having been erroneously associated with the patient. Additionally, or in the alternative, the operations 200 may include identifying a first matched patient information dataset that includes a first patient information data subset having a known association with a first patient and a first candidate patient information data subset having been associated with the first patient. The first candidate patient information data subset may have been associated with the first patient based on a first match score between the first patient information data subset and the first candidate patient information data subset. The operations 200 may include identifying a first non-healthcare dataset associated with the first patient and extracting a first set of digital identity data from the first non-healthcare dataset. The operations 200 may include computing a second match score between the first set of digital identity data and the first candidate patient information data subset. Responsive to the second match score falling below a threshold, the operations 200 may include generating a first confidence metric indicating a first confidence level of the first candidate patient information data subset having been erroneously associated with the first patient.

Additionally, or in the alternative, the operations 200 may include identifying a second matched patient information dataset that includes a second patient information data subset having a known association with a second patient and a second candidate patient information data subset having been associated with the second patient. The second candidate patient information data subset may have been associated with the second patient based on a first match score between the second patient information data subset and the second candidate patient information data subset. The operations 200 may include identifying a second non-healthcare dataset associated with the second patient and extracting a second set of digital identity data from the second non-healthcare dataset. The operations 200 may include computing a second match score between the second set of digital identity data and the second candidate patient information data subset. Responsive to the second match score meeting the threshold, the operations 200 may include generating a second confidence metric indicating a second confidence level of the second candidate patient information data subset having been correctly associated with the second patient.

With respect to identifying healthcare datasets that may have been erroneously non-matched with a patient, the operations 200 may include identifying a patient information dataset that has been identified as non-matched to a patient, and a non-healthcare dataset associated with the patient. The operations 200 may include computing a match score between the non-matched patient information dataset and the non-healthcare dataset, such as digital identity data extracted from the non-healthcare dataset. Responsive to the match score meeting a threshold, the operations 200 may include generating a confidence metric indicating a confidence level of the patient information data subset having been erroneously non-matched to the patient. Additionally, or in the alternative, the operations 200 may include identifying a first non-matched patient information dataset that includes a first patient information data subset having a known association with a first patient and a first candidate patient information data subset having been identified as non-matching to the first patient. The first candidate patient information data subset may have been identified as non-matching to the first patient based on a first match score between the first patient information data subset and the first candidate patient information data subset. The operations 200 may include identifying a first non-healthcare dataset associated with the first patient and extracting a first set of digital identity data from the first non-healthcare dataset. The operations 200 may include computing a second match score between the first set of digital identity data and the first candidate patient information data subset. Responsive to the second match score meeting a threshold, the operations 200 may include generating a first confidence metric indicating a first confidence level of the first candidate patient information data subset having been erroneously identified as non-matching to first patient.

Additionally, or in the alternative, the operations 200 may include identifying a second non-matched patient information dataset that includes a second patient information data subset having a known association with a second patient and a second candidate patient information data subset having been identified as non-matching to the second patient. The second candidate patient information data subset may have been identified as non-matching to the second patient based on a first match score between the second patient information data subset and the second candidate patient information data subset. The operations 200 may include identifying a second non-healthcare dataset associated with the second patient and extracting a second set of digital identity data from the second non-healthcare dataset. The operations 200 may include computing a second match score between the second set of digital identity data and the second candidate patient information data subset. Responsive to the second match score falling below the threshold, the operations 200 may include generating a second confidence metric indicating a second confidence level of the second candidate patient information data subset having been correctly identified as non-matching to the second patient.

In one example, the operations 200 may include generating entries in a quality assurance data structure. The entries may include a first entry that associates the first confidence metric with a first indicator identifying the first candidate patient information data subset. Additionally, or in the alternative, the entries may include a second entry that associates the second confidence metric with a second indicator identifying the second candidate patient information data subset.

### 4. PROCESSING PATIENT INFORMATION

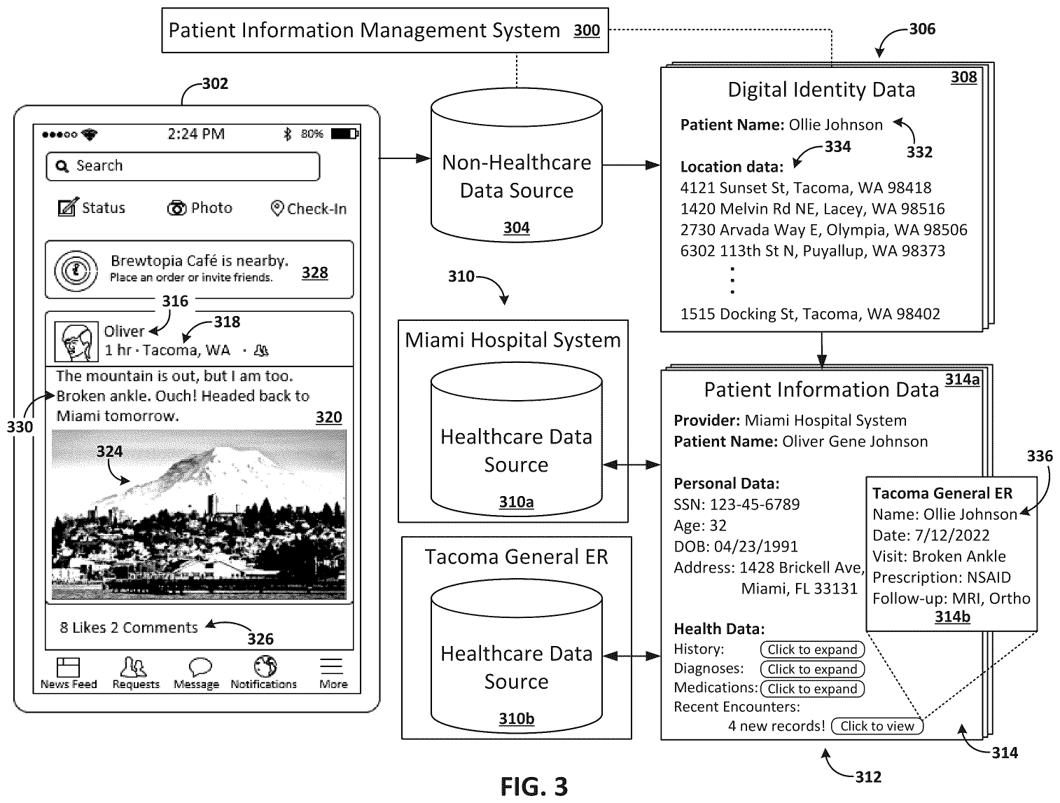

Referring now to FIG. 3, example embodiments are further described. As shown in FIG. 3, a patient information management system 300 may be utilized to process patient information data based on non-healthcare data, including identifying and matching patient information data to a patient based on non-healthcare data. The patient information management system 300 may include one or more of the features described with reference to FIG. 1. Additionally, or in the alternative, the patient information management system 300 may be configured to perform one or more of the operations described with reference to FIGS. 1, 2A, and/or 2B.

As shown, a patient associated with the patient management system 300 may generate non-healthcare data by using a user computing device 302. In one example, the user computing device 302 may include a mobile telecommunications device, as shown. The non-healthcare data generated by the user computing device 302 may be transmitted to a non-healthcare data source 304 associated with the patient information management system 300. The patient information management system 300 may obtain the non-healthcare data and store the non-healthcare data in a digital identity repository 306 in a data structure that associates the non-healthcare data with the patient. The non-healthcare data in the digital identity repository 306 may include one or more digital identity datasets 308 associated with the patient.

As further shown, the patient information management system 300 may obtain healthcare data from one or more healthcare data sources 310. The one or more healthcare data sources may be respectively associated with a different healthcare provider system and/or a different geographic location. For example, as shown, a first healthcare data source 310a is associated with a first healthcare provider system associated with a first geographic location, and a second healthcare data source 310b is associated with a second healthcare provider system associated with a second geographic location. The healthcare data may be stored in a patient information repository 312. The patient information repository 312 may include a data structure that associates the non-healthcare data with the patient. The healthcare data in the patient information repository 312 may include one or more patient information datasets 314 associated with the patient.

In one example, the non-healthcare data may include data associated with a social media platform. The non-healthcare data source 304 may include a data repository associated with the social media platform. The patient may generate non-healthcare data by interacting with the social media platform. The non-healthcare data may include the one or more digital identity datasets 308. For example, as shown, the digital identity datasets 308 may include the patient's name 316 and/or location data 318 associated with the patient. As shown, the location data 318 may indicate a geographic location, such as a city, where the patient is located. In one example, the location data 318 may be tracked by the social media platform, for example, based on geolocation data associated with the user computing device 302. Additionally, or in the alternative, geolocation data may be obtained from the user computing device 302, such as from a non-healthcare data source 304 associated with a mobile telecommunications carrier. The mobile telecommunications carrier may be utilized by the patient to provide mobile telecommunications services associated with the user computing device 302. The non-healthcare data may include geolocation data obtained from the user computing device 302 and/or from the mobile telecommunications carrier.

In one example, as shown in FIG. 3, the patient may post a status update 320 to the social media platform. Digital identity data indicative of a geographic location may be determined based on the status update 320 included in or represented by the digital identity datasets 308. For example, as shown, the status update 320 may mention a geographic location, such as in text of the status update 320. Additionally, or in the alternative, a geographic location may be inferred from a status update 320. For example, the status update 320 may mention information from which a geographic location may be inferred. The geographic location may be identified from the digital identity datasets 308 representing text in the status update 320. Additionally, or in the alternative, a geographic location may be determined based on a digital image 322, such as a digital photo. The digital image 322 may include content that is indicative of a location. For example, the digital image 322 may include a landmark 324 associated with a known location. A landmark 324 may be identified using a digital image processing algorithm. Additionally, or in the alternative, a geographic location may be determined from interactions 326, such as reactions or comments, associated with the status update 320, or from location data corresponding to other users associated with the interactions 326. Additionally, or in the alternative, a geographic location may be identified based on a combination of items of digital identity datasets 308, such as a combination of those mentioned above. Additionally, or in the alternative, a geographic location may be identified based on a location notification 328 generated by the social media platform or the user computing device 302. For example, the location notification 328 may identify a nearby geographic location.

In one example, the digital identity datasets 308 include a reference to health data. The reference to health data may be associated with a geographic location in the digital identity datasets 308. For example, as shown in FIG. 3, the status update 320 mentions a health data item 330. The patient information management system 300 may infer that healthcare data may exist at one or more healthcare data sources 310 based on digital identity datasets 308, such as the health data item 330 and/or the association between the health data item 330 and the geographic location in the digital identity datasets 308.

Referring further to FIG. 3, the non-healthcare data, including digital identity datasets 308, associated with the user computing device 302 and/or the social medial platform may be transferred to the digital identity repository 306, for example by way of the non-healthcare data source 304. The non-healthcare data in the digital identity repository 306 may include all or a portion of the digital identity datasets 308. For example, FIG. 3 shows digital identity datasets 308 that includes a known patient name 332 and geographic location data 334. Additionally, or in the alternative, the non-healthcare data in the digital identity repository 306 may include any one or more additional items of digital identity data described herein.

As shown in FIG. 3, non-healthcare data, including the digital identity datasets 308, may be utilized to process patient information. In one example, the digital identity datasets 308 may be utilized to determine a subset of healthcare data sources 310 to search for patient information datasets 314 associated with the patient. For example, one or more healthcare data sources 310 associated with a geographic location in the digital identity datasets 308 may be selected to search for patient information.

In one example, as shown, a first one or more patient information datasets 314a may have a known association with the patient. Additionally, or in the alternative, the first one or more patient information datasets 314a may be identified by the patient information management system 300 based on the non-healthcare data, such as the one or more digital identity datasets 308. For example, the status update 320 included in the digital identity datasets 308 mentions a geographic location associated with the first healthcare data source 310a. The first one or more patient information datasets 314a may be identified based at least in part on the geographic location associated with the first healthcare data source 310a mentioned in the status update 320.

Additionally, or in the alternative, a second one or more patient information datasets 314b may be unassociated with the patient. For example, the second one or more patient information datasets 314b may be associated with an unknown patient. The second one or more patient information datasets 314b may be identified by the patient information management system 300 based on the non-healthcare data, such as the one or more digital identity datasets 308. For example, as shown, one or more healthcare data sources 310 may be searched based on the geographic location data 334 in the digital identity datasets 308. The second one or more patient information datasets 314b may be identified based on the search. As shown, the geographic location data 334 in the digital identity datasets 308 corresponds to a geographic location associated with the second healthcare data source 310b. The second one or more patient information datasets 314b may be obtained from the second healthcare data source 310b.

In one example, the digital identity datasets 308 may be utilized to identify and match patient information datasets with the patient. As shown in FIG. 3, the second one or more patient information datasets 314b may include personal data, such as an unmatched patient name 336. The personal data, such as the unmatched patient name 336, in the second one or more patient information datasets 314b may be matched to digital identity data, such as the known patient name 332 in the one or more digital identity datasets 308. Based on the match between the digital identity datasets 308 and the second one or more patient information datasets 314b (e.g., the match between the known patient name 332 and the unmatched patient name 336), the second one or more patient information datasets 314b may be associated with the patient and/or the first one or more patient information datasets 314b. The second one or more patient information datasets 314b, having been matched with the patient, may be stored in a data structure, such as in the patient information repository 312, that associates the second one or more patient information datasets 314b with the patient and/or with the first one or more patient information datasets 314a associated with the patient. Additionally, or in the alternative, the first one or more patient information datasets 314a may be updated based on the second one or more patient information datasets 314b. Additionally, or in the alternative, the second one or more patient information datasets 314b, having been matched with the patient, may be transmitted to one or more healthcare provider systems, such as the healthcare provider system associated with the first healthcare data source 310a. The second one or more patient information datasets 314b may be stored in a data structure, such as in the first healthcare data source 310a, that associates the second one or more patient information datasets 314b with the patient and/or with the first one or more patient information datasets 314a associated with the patient.

### 5. PRACTICAL APPLICATIONS, ADVANTAGES & IMPROVEMENTS

The presently disclosed systems and methods provide improved matching of healthcare data to patients. By utilizing non-healthcare data, the presently disclosed systems and methods may identify healthcare data associated with a patient that may otherwise go unidentified. Additionally, or in the alternative, the presently disclosed systems and methods may more expediently identify healthcare data associated with a patient. Further, the presently disclosed systems and methods may reduce data searching and processing costs, including reduced computing resources utilized to search for healthcare records. Further, the presently disclosed systems and methods may improve patient-matching accuracy, for example, by providing a non-healthcare data source as a basis for comparison. The non-healthcare data source may be utilized to catch errors that may otherwise go unnoticed, such as errors that have propagated across healthcare datasets.

The improved matching of healthcare data to patients may lead to better healthcare treatments and outcomes for patients. For example, by having access to a more comprehensive and accurate healthcare dataset associated with a patient, healthcare providers can better ensure that they are providing the right treatments. Further, the improved patient-matching provided by the present disclosure may lead to greater interoperability between different healthcare provider systems, improved exchange of healthcare data between different healthcare provider systems, and better coordination of healthcare treatments and improved patient outcomes.

### 6. COMPUTER NETWORKS AND CLOUD NETWORKS

In one or more embodiments, a computer network provides connectivity among a set of nodes. The nodes may be local to and/or remote from each other. The nodes are connected by a set of links. Examples of links include a coaxial cable, an unshielded twisted cable, a copper cable, an optical fiber, and a virtual link.

A subset of nodes implements the computer network. Examples of such nodes include a switch, a router, a firewall, and a network address translator (NAT). Another subset of nodes uses the computer network. Such nodes (also referred to as “hosts”) may execute a client process and/or a server process. A client process makes a request for a computing service (such as, execution of a particular application, and/or storage of a particular amount of data). A server process responds by executing the requested service and/or returning corresponding data.

A computer network may be a physical network, including physical nodes connected by physical links. A physical node is any digital device. A physical node may be a function-specific hardware device, such as a hardware switch, a hardware router, a hardware firewall, and a hardware NAT. Additionally or alternatively, a physical node may be a generic machine that is configured to execute various virtual machines and/or applications performing respective functions. A physical link is a physical medium connecting two or more physical nodes. Examples of links include a coaxial cable, an unshielded twisted cable, a copper cable, and an optical fiber.

A computer network may be an overlay network. An overlay network is a logical network implemented on top of another network (such as, a physical network). Each node in an overlay network corresponds to a respective node in the underlying network. Hence, each node in an overlay network is associated with both an overlay address (to address to the overlay node) and an underlay address (to address the underlay node that implements the overlay node). An overlay node may be a digital device and/or a software process (such as, a virtual machine, an application instance, or a thread) A link that connects overlay nodes is implemented as a tunnel through the underlying network. The overlay nodes at either end of the tunnel treat the underlying multi-hop path between them as a single logical link. Tunneling is performed through encapsulation and decapsulation.

In an embodiment, a client may be local to and/or remote from a computer network. The client may access the computer network over other computer networks, such as a private network or the Internet. The client may communicate requests to the computer network using a communications protocol, such as Hypertext Transfer Protocol (HTTP). The requests are communicated through an interface, such as a client interface (such as a web browser), a program interface, or an application programming interface (API).

In an embodiment, a computer network provides connectivity between clients and network resources. Network resources include hardware and/or software configured to execute server processes. Examples of network resources include a processor, a data storage, a virtual machine, a container, and/or a software application. Network resources are shared amongst multiple clients. Clients request computing services from a computer network independently of each other. Network resources are dynamically assigned to the requests and/or clients on an on-demand basis. Network resources assigned to each request and/or client may be scaled up or down based on, for example, (a) the computing services requested by a particular client, (b) the aggregated computing services requested by a particular tenant, and/or (c) the aggregated computing services requested of the computer network. Such a computer network may be referred to as a “cloud network.”

In an embodiment, a service provider provides a cloud network to one or more end users. Various service models may be implemented by the cloud network, including but not limited to Software-as-a-Service (SaaS), Platform-as-a-Service (PaaS), and Infrastructure-as-a-Service (IaaS). In SaaS, a service provider provides end users the capability to use the service provider's applications, which are executing on the network resources. In PaaS, the service provider provides end users the capability to deploy custom applications onto the network resources. The custom applications may be created using programming languages, libraries, services, and tools supported by the service provider. In IaaS, the service provider provides end users the capability to provision processing, storage, networks, and other fundamental computing resources provided by the network resources. Any arbitrary applications, including an operating system, may be deployed on the network resources.

In an embodiment, various deployment models may be implemented by a computer network, including but not limited to a private cloud, a public cloud, and a hybrid cloud. In a private cloud, network resources are provisioned for exclusive use by a particular group of one or more entities (the term “entity” as used herein refers to a corporation, organization, person, or other entity). The network resources may be local to and/or remote from the premises of the particular group of entities. In a public cloud, cloud resources are provisioned for multiple entities that are independent from each other (also referred to as “tenants” or “customers”). The computer network and the network resources thereof are accessed by clients corresponding to different tenants. Such a computer network may be referred to as a “multi-tenant computer network.” Several tenants may use a same particular network resource at different times and/or at the same time. The network resources may be local to and/or remote from the premises of the tenants. In a hybrid cloud, a computer network comprises a private cloud and a public cloud. An interface between the private cloud and the public cloud allows for data and application portability. Data stored at the private cloud and data stored at the public cloud may be exchanged through the interface. Applications implemented at the private cloud and applications implemented at the public cloud may have dependencies on each other. A call from an application at the private cloud to an application at the public cloud (and vice versa) may be executed through the interface.

In an embodiment, tenants of a multi-tenant computer network are independent of each other. For example, a business or operation of one tenant may be separate from a business or operation of another tenant. Different tenants may demand different network requirements for the computer network. Examples of network requirements include processing speed, amount of data storage, security requirements, performance requirements, throughput requirements, latency requirements, resiliency requirements, Quality of Service (QOS) requirements, tenant isolation, and/or consistency. The same computer network may need to implement different network requirements demanded by different tenants.

In one or more embodiments, in a multi-tenant computer network, tenant isolation is implemented to ensure that the applications and/or data of different tenants are not shared with each other. Various tenant isolation approaches may be used.

In an embodiment, each tenant is associated with a tenant ID. Each network resource of the multi-tenant computer network is tagged with a tenant ID. A tenant is permitted access to a particular network resource only if the tenant and the particular network resources are associated with a same tenant ID.

In an embodiment, each tenant is associated with a tenant ID. Each application, implemented by the computer network, is tagged with a tenant ID. Additionally or alternatively, each data structure and/or dataset, stored by the computer network, is tagged with a tenant ID. A tenant is permitted access to a particular application, data structure, and/or dataset only if the tenant and the particular application, data structure, and/or dataset are associated with a same tenant ID.

As an example, each database implemented by a multi-tenant computer network may be tagged with a tenant ID. Only a tenant associated with the corresponding tenant ID may access data of a particular database. As another example, each entry in a database implemented by a multi-tenant computer network may be tagged with a tenant ID. Only a tenant associated with the corresponding tenant ID may access data of a particular entry. However, the database may be shared by multiple tenants.

In an embodiment, a subscription list indicates which tenants have authorization to access which applications. For each application, a list of tenant IDs of tenants authorized to access the application is stored. A tenant is permitted access to a particular application only if the tenant ID of the tenant is included in the subscription list corresponding to the particular application.

In an embodiment, network resources (such as digital devices, virtual machines, application instances, and threads) corresponding to different tenants are isolated to tenant-specific overlay networks maintained by the multi-tenant computer network. As an example, packets from any source device in a tenant overlay network may only be transmitted to other devices within the same tenant overlay network. Encapsulation tunnels are used to prohibit any transmissions from a source device on a tenant overlay network to devices in other tenant overlay networks. Specifically, the packets received from the source device are encapsulated within an outer packet. The outer packet is transmitted from a first encapsulation tunnel endpoint (in communication with the source device in the tenant overlay network) to a second encapsulation tunnel endpoint (in communication with the destination device in the tenant overlay network). The second encapsulation tunnel endpoint decapsulates the outer packet to obtain the original packet transmitted by the source device. The original packet is transmitted from the second encapsulation tunnel endpoint to the destination device in the same particular overlay network.

### 7. MICROSERVICE APPLICATIONS

According to one or more embodiments, the techniques described herein are implemented in a microservice architecture. A microservice in this context refers to software logic designed to be independently deployable, having endpoints that may be logically coupled to other microservices to build a variety of applications. Applications built using microservices are distinct from monolithic applications, which are designed as a single fixed unit and generally comprise a single logical executable. With microservice applications, different microservices are independently deployable as separate executables. Microservices may communicate using HyperText Transfer Protocol (HTTP) messages and/or according to other communication protocols via API endpoints. Microservices may be managed and updated separately, written in different languages, and be executed independently from other microservices.

Microservices provide flexibility in managing and building applications. Different applications may be built by connecting different sets of microservices without changing the source code of the microservices. Thus, the microservices act as logical building blocks that may be arranged in a variety of ways to build different applications. Microservices may provide monitoring services that notify a microservices manager (such as If-This-Then-That (IFTTT), Zapier, or Oracle Self-Service Automation (OSSA)) when trigger events from a set of trigger events exposed to the microservices manager occur. Microservices exposed for an application may alternatively or additionally provide action services that perform an action in the application (controllable and configurable via the microservices manager by passing in values, connecting the actions to other triggers and/or data passed along from other actions in the microservices manager) based on data received from the microservices manager. The microservice triggers and/or actions may be chained together to form recipes of actions that occur in optionally different applications that are otherwise unaware of or have no control or dependency on each other. These managed applications may be authenticated or plugged in to the microservices manager, for example, with user-supplied application credentials to the manager, without requiring reauthentication each time the managed application is used alone or in combination with other applications.

In one or more embodiments, microservices may be connected via a GUI. For example, microservices may be displayed as logical blocks within a window, frame, other element of a GUI. A user may drag and drop microservices into an area of the GUI used to build an application. The user may connect the output of one microservice into the input of another microservice using directed arrows or any other GUI element. The application builder may run verification tests to confirm that the output and inputs are compatible (e.g., by checking the datatypes, size restrictions, etc.)

### Triggers

The techniques described above may be encapsulated into a microservice, according to one or more embodiments. In other words, a microservice may trigger a notification (into the microservices manager for optional use by other plugged in applications, herein referred to as the “target” microservice) based on the above techniques and/or may be represented as a GUI block and connected to one or more other microservices. The trigger condition may include absolute or relative thresholds for values, and/or absolute or relative thresholds for the amount or duration of data to analyze, such that the trigger to the microservices manager occurs whenever a plugged-in microservice application detects that a threshold is crossed. For example, a user may request a trigger into the microservices manager when the microservice application detects a value has crossed a triggering threshold.

In one embodiment, the trigger, when satisfied, might output data for consumption by the target microservice. In another embodiment, the trigger, when satisfied, outputs a binary value indicating the trigger has been satisfied, or outputs the name of the field or other context information for which the trigger condition was satisfied. Additionally or alternatively, the target microservice may be connected to one or more other microservices such that an alert is input to the other microservices. Other microservices may perform responsive actions based on the above techniques, including, but not limited to, deploying additional resources, adjusting system configurations, and/or generating GUIs.

### Actions

In one or more embodiments, a plugged-in microservice application may expose actions to the microservices manager. The exposed actions may receive, as input, data or an identification of a data object or location of data, that causes data to be moved into a data cloud.

In one or more embodiments, the exposed actions may receive, as input, a request to increase or decrease existing alert thresholds. The input might identify existing in-application alert thresholds and whether to increase or decrease, or delete the threshold. Additionally, or alternatively, the input might request the microservice application to create new in-application alert thresholds. The in-application alerts may trigger alerts to the user while logged into the application, or may trigger alerts to the user using default or user-selected alert mechanisms available within the microservice application itself, rather than through other applications plugged into the microservices manager.

In one or more embodiments, the microservice application may generate and provide an output based on input that identifies, locates, or provides historical data, and defines the extent or scope of the requested output. The action, when triggered, causes the microservice application to provide, store, or display the output, for example, as a data model or as aggregate data that describes a data model.

### 10. HARDWARE OVERVIEW

According to one embodiment, the techniques described herein are implemented by one or more special-purpose computing devices. The special-purpose computing devices may be hard-wired to perform the techniques, or may include digital electronic devices such as one or more application-specific integrated circuits (ASICs), field programmable gate arrays (FPGAs), or network processing units (NPUs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, FPGAs, or NPUs with custom programming to accomplish the techniques. The special-purpose computing devices may be desktop computer systems, portable computer systems, handheld devices, networking devices or any other device that incorporates hard-wired and/or program logic to implement the techniques.

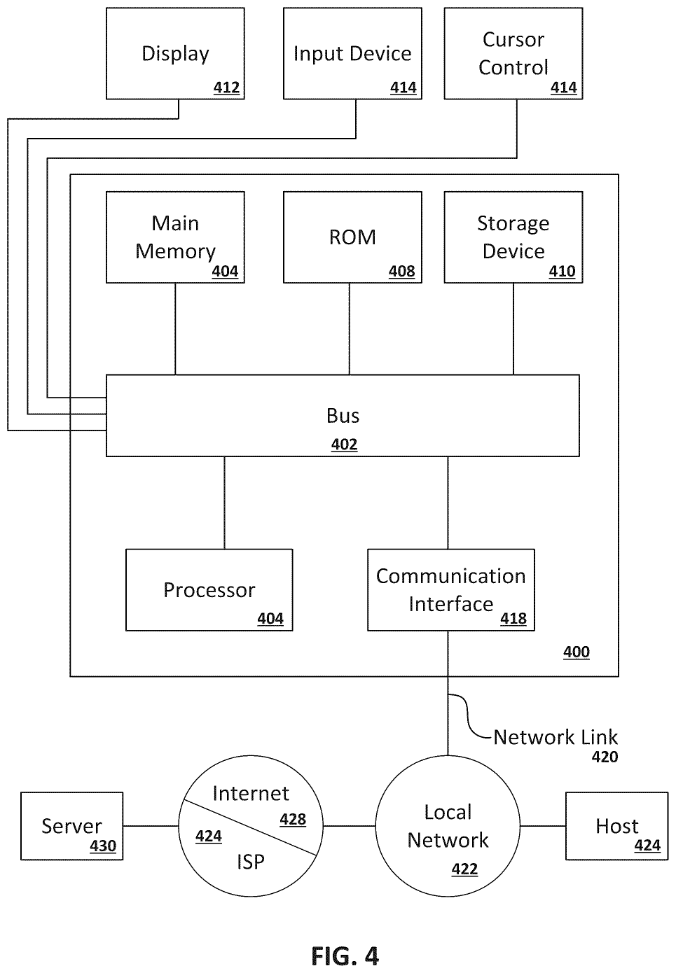

For example, FIG. 4 is a block diagram that illustrates a computer system 400 upon which an embodiment of the invention may be implemented. Computer system 400 includes a bus 402 or other communication mechanism for communicating information, and a hardware processor 404 coupled with bus 402 for processing information. Hardware processor 404 may be, for example, a general-purpose microprocessor.

Computer system 400 also includes a main memory 406, such as a random-access memory (RAM) or other dynamic storage device, coupled to bus 402 for storing information and instructions to be executed by processor 404. Main memory 406 also may be used for storing temporary variables or other intermediate information during execution of instructions to be executed by processor 404. Such instructions, when stored in non-transitory storage media accessible to processor 404, render computer system 400 into a special-purpose machine that is customized to perform the operations specified in the instructions.

Computer system 400 further includes a read only memory (ROM) 408 or other static storage device coupled to bus 402 for storing static information and instructions for processor 404. A storage device 410, such as a magnetic disk or optical disk, is provided and coupled to bus 402 for storing information and instructions.

Computer system 400 may be coupled via bus 402 to a display 412, such as a cathode ray tube (CRT), for displaying information to a computer user. An input device 414, including alphanumeric and other keys, is coupled to bus 402 for communicating information and command selections to processor 404. Another type of user input device is cursor control 416, such as a mouse, a trackball, or cursor direction keys for communicating direction information and command selections to processor 404 and for controlling cursor movement on display 412. This input device typically has two degrees of freedom in two axes, a first axis (e.g., x) and a second axis (e.g., y), that allows the device to specify positions in a plane.

Computer system 400 may implement the techniques described herein using customized hard-wired logic, one or more ASICs or FPGAs, firmware and/or program logic which in combination with the computer system causes or programs computer system 400 to be a special-purpose machine. According to one embodiment, the techniques herein are performed by computer system 400 in response to processor 404 executing one or more sequences of one or more instructions contained in main memory 406. Such instructions may be read into main memory 406 from another storage medium, such as storage device 410. Execution of the sequences of instructions contained in main memory 406 causes processor 404 to perform the process steps described herein. In alternative embodiments, hard-wired circuitry may be used in place of or in combination with software instructions.

The term “storage media” as used herein refers to any non-transitory media that store data and/or instructions that cause a machine to operate in a specific fashion. Such storage media may comprise non-volatile media and/or volatile media. Non-volatile media includes, for example, optical or magnetic disks, such as storage device 410. Volatile media includes dynamic memory, such as main memory 406. Common forms of storage media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, content-addressable memory (CAM), and ternary content-addressable memory (TCAM).

Storage media is distinct from but may be used in conjunction with transmission media. Transmission media participates in transferring information between storage media. For example, transmission media includes coaxial cables, copper wire and fiber optics, including the wires that comprise bus 402. Transmission media can also take the form of acoustic or light waves, such as those generated during radio-wave and infra-red data communications.

Various forms of media may be involved in carrying one or more sequences of one or more instructions to processor 404 for execution. For example, the instructions may initially be carried on a magnetic disk or solid-state drive of a remote computer. The remote computer can load the instructions into its dynamic memory and send the instructions over a telephone line using a modem. A modem local to computer system 400 can receive the data on the telephone line and use an infra-red transmitter to convert the data to an infra-red signal. An infra-red detector can receive the data carried in the infra-red signal and appropriate circuitry can place the data on bus 402. Bus 402 carries the data to main memory 406, from which processor 404 retrieves and executes the instructions. The instructions received by main memory 406 may optionally be stored on storage device 410 either before or after execution by processor 404.

Computer system 400 also includes a communication interface 418 coupled to bus 402. Communication interface 418 provides a two-way data communication coupling to a network link 420 that is connected to a local network 422. For example, communication interface 418 may be an integrated services digital network (ISDN) card, cable modem, satellite modem, or a modem to provide a data communication connection to a corresponding type of telephone line. As another example, communication interface 418 may be a local area network (LAN) card to provide a data communication connection to a compatible LAN. Wireless links may also be implemented. In any such implementation, communication interface 418 sends and receives electrical, electromagnetic, or optical signals that carry digital data streams representing various types of information.

Network link 420 typically provides data communication through one or more networks to other data devices. For example, network link 420 may provide a connection through local network 422 to a host computer 424 or to data equipment operated by an Internet Service Provider (ISP) 426. ISP 426 in turn provides data communication services through the world wide packet data communication network now commonly referred to as the “Internet” 428. Local network 422 and Internet 428 both use electrical, electromagnetic, or optical signals that carry digital data streams. The signals through the various networks and the signals on network link 420 and through communication interface 418, which carry the digital data to and from computer system 400, are example forms of transmission media.

Computer system 400 can send messages and receive data, including program code, through the network(s), network link 420 and communication interface 418. In the Internet example, a server 430 might transmit a requested code for an application program through Internet 428, ISP 426, local network 422 and communication interface 418.

The received code may be executed by processor 404 as it is received, and/or stored in storage device 410, or other non-volatile storage for later execution.

### 8. MISCELLANEOUS; EXTENSIONS

Embodiments are directed to a system with one or more devices that include a hardware processor and that are configured to perform any of the operations described herein and/or recited in any of the claims below.

In an embodiment, a non-transitory computer readable storage medium comprises instructions which, when executed by one or more hardware processors, causes performance of any of the operations described herein and/or recited in any of the claims.

Any combination of the features and functionalities described herein may be used in accordance with one or more embodiments. In the foregoing specification, embodiments have been described with reference to numerous specific details that may vary from implementation to implementation. The specification and drawings are, accordingly, to be regarded in an illustrative rather than a restrictive sense. The sole and exclusive indicator of the scope of the invention, and what is intended by the applicants to be the scope of the invention, is the literal and equivalent scope of the set of claims that issue from this application, in the specific form in which such claims issue, including any subsequent correction.

What is claimed is:

1. A non-transitory computer readable medium comprising instructions which, when executed by one or more hardware processors, causes performance of operations comprising:
identifying a first non-healthcare data source associated with a first patient;
extracting, from the first non-healthcare data source, a non-healthcare dataset corresponding to the first patient;
selecting, based at least on the non-healthcare dataset corresponding to the first patient, a target subset of healthcare data from a set of healthcare data;
executing a query on the target subset of healthcare data to identify a first candidate patient information dataset associated with the first patient;
creating or updating a patient information data structure based on the first candidate patient information dataset associated with the first patient.

2. The medium of claim 1, wherein the operations further comprise:
subsequent to identifying the first candidate patient information dataset, computing a match score between (a) known patient information dataset associated with the first patient and (b) the first candidate patient information dataset;
determining that the match score meets a threshold;
wherein creating or updating the patient information data structure based on the first candidate patient information dataset is responsive at least to determining that the match score meets the threshold.

3. The medium of claim 1, wherein selecting the target subset of healthcare data from the set of healthcare data based at least on the non-healthcare dataset corresponding to the first patient comprises:
extracting digital identity data, corresponding to the first patient, from the non-healthcare dataset corresponding to the first patient;
selecting the target subset of healthcare data based on the digital identity data corresponding to the first patient.

4. The medium of claim 1, wherein the operations further comprise:
identifying a second candidate patient information dataset associated with the first patient, wherein the first candidate patient information dataset corresponds to a first healthcare data source and the second candidate patient information dataset corresponding to a second healthcare data source; and
associating the second candidate patient information dataset with the first candidate patient information dataset in the patient information data structure.

5. The medium of claim 4, wherein the operations further comprise:
augmenting the second candidate patient information dataset based on at least a portion of the first candidate patient information dataset.

6. The medium of claim 1,
wherein the target subset of healthcare data is associated with a first healthcare provider system; and
wherein the operations further comprise:
identifying a second candidate patient information dataset associated with the first patient, the second candidate patient information dataset corresponding to a second healthcare provider system; and
transmitting to the second healthcare provider system, at least one of: (a) an indication of the first candidate patient information dataset having been associated with the first patient; or (b) the first candidate patient information dataset.

7. The medium of claim 6, wherein the operations further comprise:
identifying a first patient name in the first candidate patient information dataset, wherein the first healthcare provider system corresponds to a first geographic location;
identifying a second patient name in the second candidate patient information dataset, wherein the second healthcare provider system corresponds to a second geographic location;
determining, based on the non-healthcare dataset, that the first patient name and the second patient name each correspond to the first patient, wherein the first patient name differs from the second patient name.

8. The medium of claim 1, wherein selecting the target subset of healthcare data from the set of healthcare data comprises:
executing a second query on the set of healthcare data to identify a candidate subset of healthcare data corresponding to a geographic location indicated based on the non-healthcare dataset;
selecting the candidate subset of healthcare data as the target subset of healthcare data.

9. The medium of claim 1, wherein selecting the target subset of healthcare data from the set of healthcare data comprises:
extracting digital identity data indicative of a geographic location from the non-healthcare dataset;
executing a second query on a set of healthcare data sources to identify a target subset of healthcare data sources corresponding to the geographic location, the target subset of healthcare data sources comprising at least one healthcare data source;
identifying a candidate set of healthcare data from the target subset of healthcare data sources;
selecting the candidate set of healthcare data as the set of healthcare data; and
selecting the target subset of healthcare data from the set of healthcare data.

10. The medium of claim 1, wherein the operations further comprise:
extracting digital identity data from the non-healthcare dataset, the digital identity data comprising at least one personal data associated with the first patient, and
configuring the query based on the at least one personal data.

11. The medium of claim 1,
wherein the query identifies the first candidate patient information dataset and a second candidate patient information dataset, and
wherein the operations further comprise:
computing a first match score between the non-healthcare dataset and the first candidate patient information dataset, and selecting the first candidate patient information dataset responsive to the first match score meeting a threshold; and
computing a second match score between the non-healthcare dataset and the second candidate patient information dataset, and refraining from selecting the second candidate patient information dataset responsive to the second match score falling below the threshold.

12. The medium of claim 1, wherein the operations further comprise:
identifying a second non-healthcare dataset associated with the first patient;
computing a match score between the second non-healthcare dataset and the first candidate patient information dataset;
responsive to the match score meeting a threshold, updating the patient information data structure to indicate that the first candidate patient information dataset comprises patient information corresponding to the first patient.

13. A non-transitory computer readable medium comprising instructions which, when executed by one or more hardware processors, causes performance of operations comprising:
identifying a non-healthcare dataset associated with a first patient;
identifying a first candidate patient information dataset;
computing a match score between the non-healthcare dataset and the first candidate patient information dataset;
responsive to the match score meeting a threshold, selecting the first candidate patient information dataset as a first patient information dataset; and
storing the first patient information dataset in a patient information data structure, wherein the patient information data structure associates the first patient information dataset with the first patient.

14. The medium of claim 13, wherein computing the match score between the non-healthcare dataset and the first candidate patient information dataset comprises:
extracting digital identity data, corresponding to the first patient, from the non-healthcare dataset corresponding to the first patient;
computing the match score between the digital identity data and the first candidate patient information dataset.

15. The medium of claim 13, wherein the operations further comprise:
extracting digital identity data, corresponding to the first patient, from the non-healthcare dataset corresponding to the first patient;
augmenting the first patient information dataset based on the digital identity data to generate a first augmented patient information dataset; and
storing the first augmented patient information dataset in the patient information data structure, wherein the patient information data structure associates the first augmented patient information dataset with the first patient.

16. The medium of claim 13, wherein the operations further comprise:
extracting a first set of patient information data from the first patient information dataset;
identifying a second patient information dataset associated with the first patient;
augmenting the second patient information dataset based on the first set of patient information data to generate a second augmented patient information dataset; and
storing the second augmented patient information dataset in the patient information data structure, wherein the patient information data structure associates the second augmented patient information dataset with the first patient.

17. The medium of claim 13, wherein identifying the non-healthcare dataset associated with the first patient comprises:
extracting a first set of patient information data from a known patient information dataset associated with the first patient;
executing a query on a group of non-healthcare datasets, wherein the query is based on the first set of patient information data; and
receiving a first query result identifying a candidate non-healthcare dataset;
computing a second match score between the candidate non-healthcare dataset and the known patient information dataset; and
responsive to the second match score meeting a second threshold, selecting the candidate non-healthcare dataset as the non-healthcare dataset associated with the first patient.

18. The medium of claim 13, wherein identifying the first candidate patient information dataset comprises:
extracting a first set of digital identity data from a known digital identity dataset associated with the first patient;
executing a query on a group of healthcare datasets, wherein the query is based on the first set of digital identity data;
receiving a query result identifying a subset of healthcare data comprising a plurality of patient information datasets including the first candidate patient information dataset;
computing a second match score between the known digital identity dataset and at least some of the plurality of patient information datasets; and
selecting the first candidate patient information dataset from the plurality of patient information datasets responsive to the second match score meeting a second threshold with respect to the first candidate patient information dataset.

19. The medium of claim 18, wherein identifying the first candidate patient information dataset comprises:
refraining from selecting as the first candidate patient information dataset, a second candidate patient information dataset from the plurality of patient information datasets responsive to the second match score falling below the second threshold with respect to the second candidate patient information dataset.

20. The medium of claim 13, wherein the operations further comprise:
responsive to the match score meeting the threshold, transmitting to at least one healthcare provider system, at least one of:
a first indication indicative of the match score meeting the threshold;
a second indication indicative of the first patient information dataset having been associated with the first patient, or
an augmented patient information dataset associated with the first patient, the augmented patient information dataset having been generated based at least in part on digital identity data extracted from the non-healthcare dataset.

21. The medium of claim 13, wherein the operations further comprise:
identifying a second candidate patient information dataset;
computing a second match score between the non-healthcare dataset associated with the first patient and the second candidate patient information dataset;
responsive to the second match score falling below the threshold, creating or updating the patient information data structure to include an indication that the second candidate patient information dataset is unassociated with the first patient.

22. The medium of claim 21, wherein the operations further comprise:
responsive to the second match score falling below the threshold, transmitting to at least one healthcare provider system, at least one of:
a first indication indicative of the second match score falling below the threshold;
a second indication indicative of the first patient information dataset being unassociated with the first patient, or
an augmented patient information dataset unassociated with the first patient.

23. The medium of claim 13, wherein the operations further comprise:
identifying a second candidate patient information dataset;
computing a second match score between the second candidate patient information dataset and the first candidate patient information dataset;
responsive to the second match score meeting a second threshold, selecting the second candidate patient information dataset as a second patient information dataset; and
storing the second patient information dataset in the patient information data structure, wherein the patient information data structure associates the second patient information dataset with the first patient.

24. A method, comprising:
identifying a first non-healthcare data source associated with a first patient;
extracting, from the first non-healthcare data source, a non-healthcare dataset corresponding to the first patient;
selecting, based at least on the non-healthcare dataset corresponding to the first patient, a target subset of healthcare data from a set of healthcare data;
executing a query on the target subset of healthcare data to identify a first candidate patient information dataset associated with the first patient;
creating or updating a patient information data structure based on the first candidate patient information dataset associated with the first patient;
wherein the method is performed by at least one device including a hardware processor.

25. The method of claim 24, further comprising:
subsequent to identifying the first candidate patient information dataset, computing a match score between (a) known patient information dataset associated with the first patient and (b) the first candidate patient information dataset;
determining that the match score meets a threshold;
wherein creating or updating the patient information data structure based on the first candidate patient information dataset is responsive at least to determining that the match score meets the threshold.

26. The method of claim 24, wherein selecting the target subset of healthcare data from the set of healthcare data based at least on the non-healthcare dataset corresponding to the first patient comprises:
extracting digital identity data, corresponding to the first patient, from the non-healthcare dataset corresponding to the first patient;
selecting the target subset of healthcare data based on the digital identity data corresponding to the first patient.

27. The method of claim 24, further comprising:
identifying a second candidate patient information dataset associated with the first patient, wherein the first candidate patient information dataset corresponds to a first healthcare data source and the second candidate patient information dataset corresponding to a second healthcare data source; and
associating the second candidate patient information dataset with the first candidate patient information dataset in the patient information data structure.

28. The method of claim 27, further comprising:
augmenting the second candidate patient information dataset based on at least a portion of the first candidate patient information dataset.

29. The method of claim 24,
wherein the target subset of healthcare data is associated with a first healthcare provider system; and
wherein the method further comprises:
identifying a second candidate patient information dataset associated with the first patient, the second candidate patient information dataset corresponding to a second healthcare provider system; and
transmitting to the second healthcare provider system, at least one of: (a) an indication of the first candidate patient information dataset having been associated with the first patient; or (b) the first candidate patient information dataset.

30. The method of claim 29, further comprising:
identifying a first patient name in the first candidate patient information dataset, wherein the first healthcare provider system corresponds to a first geographic location;
identifying a second patient name in the second candidate patient information dataset, wherein the second healthcare provider system corresponds to a second geographic location;
determining, based on the non-healthcare dataset, that the first patient name and the second patient name each correspond to the first patient, wherein the first patient name differs from the second patient name.

31. The method of claim 24, wherein selecting the target subset of healthcare data from the set of healthcare data comprises:
executing a second query on the set of healthcare data to identify a candidate subset of healthcare data corresponding to a geographic location indicated based on the non-healthcare dataset;
selecting the candidate subset of healthcare data as the target subset of healthcare data.

32. The method of claim 24, wherein selecting the target subset of healthcare data from the set of healthcare data comprises:
extracting digital identity data indicative of a geographic location from the non-healthcare dataset;
executing a second query on a set of healthcare data sources to identify a target subset of healthcare data sources corresponding to the geographic location, the target subset of healthcare data sources comprising at least one healthcare data source;
identifying a candidate set of healthcare data from the target subset of healthcare data sources;
selecting the candidate set of healthcare data as the set of healthcare data; and
selecting the target subset of healthcare data from the set of healthcare data.

33. The method of claim 24, further comprising:
extracting digital identity data from the non-healthcare dataset, the digital identity data comprising at least one personal data associated with the first patient, and
configuring the query based on the at least one personal data.

34. The method of claim 24,
wherein the query identifies the first candidate patient information dataset and a second candidate patient information dataset, and
wherein the method further comprises:
computing a first match score between the non-healthcare dataset and the first candidate patient information dataset, and selecting the first candidate patient information dataset responsive to the first match score meeting a threshold; and
computing a second match score between the non-healthcare dataset and the second candidate patient information dataset, and refraining from selecting the second candidate patient information dataset responsive to the second match score falling below the threshold.

35. The method of claim 24, further comprising:
identifying a second non-healthcare dataset associated with the first patient;
computing a match score between the second non-healthcare dataset and the first candidate patient information dataset;
responsive to the match score meeting a threshold, updating the patient information data structure to indicate that the first candidate patient information dataset comprises patient information corresponding to the first patient.

36. A method, comprising:
identifying a non-healthcare dataset associated with a first patient;
identifying a first candidate patient information dataset;
computing a match score between the non-healthcare dataset and the first candidate patient information dataset;
responsive to the match score meeting a threshold, selecting the first candidate patient information dataset as a first patient information dataset; and
storing the first patient information dataset in a patient information data structure, wherein the patient information data structure associates the first patient information dataset with the first patient;
wherein the method is performed by at least one device including a hardware processor.

37. The method of claim 36, wherein computing the match score between the non-healthcare dataset and the first candidate patient information dataset comprises:
extracting digital identity data, corresponding to the first patient, from the non-healthcare dataset corresponding to the first patient;
computing the match score between the digital identity data and the first candidate patient information dataset.

38. The method of claim 36, further comprising:
extracting digital identity data, corresponding to the first patient, from the non-healthcare dataset corresponding to the first patient;
augmenting the first patient information dataset based on the digital identity data to generate a first augmented patient information dataset; and
storing the first augmented patient information dataset in the patient information data structure, wherein the patient information data structure associates the first augmented patient information dataset with the first patient.

39. The method of claim 36, further comprising:
extracting a first set of patient information data from the first patient information dataset;
identifying a second patient information dataset associated with the first patient;
augmenting the second patient information dataset based on the first set of patient information data to generate a second augmented patient information dataset; and
storing the second augmented patient information dataset in the patient information data structure, wherein the patient information data structure associates the second augmented patient information dataset with the first patient.

40. The method of claim 36, wherein identifying the non-healthcare dataset associated with the first patient comprises:
extracting a first set of patient information data from a known patient information dataset associated with the first patient;
executing a query on a group of non-healthcare datasets, wherein the query is based on the first set of patient information data; and
receiving a first query result identifying a candidate non-healthcare dataset;
computing a second match score between the candidate non-healthcare dataset and the known patient information dataset; and
responsive to the second match score meeting a second threshold, selecting the candidate non-healthcare dataset as the non-healthcare dataset associated with the first patient.

41. The method of claim 36, wherein identifying the first candidate patient information dataset comprises:
extracting a first set of digital identity data from a known digital identity dataset associated with the first patient;
executing a query on a group of healthcare datasets, wherein the query is based on the first set of digital identity data;
receiving a query result identifying a subset of healthcare data comprising a plurality of patient information datasets including the first candidate patient information dataset;
computing a second match score between the known digital identity dataset and at least some of the plurality of patient information datasets; and
selecting the first candidate patient information dataset from the plurality of patient information datasets responsive to the second match score meeting a second threshold with respect to the first candidate patient information dataset.

42. The method of claim 41, wherein identifying the first candidate patient information dataset comprises:
refraining from selecting as the first candidate patient information dataset, a second candidate patient information dataset from the plurality of patient information datasets responsive to the second match score falling below the second threshold with respect to the second candidate patient information dataset.

43. The method of claim 36, further comprising:
responsive to the match score meeting the threshold, transmitting to at least one healthcare provider system, at least one of:
a first indication indicative of the match score meeting the threshold;
a second indication indicative of the first patient information dataset having been associated with the first patient, or
an augmented patient information dataset associated with the first patient, the augmented patient information dataset having been generated based at least in part on digital identity data extracted from the non-healthcare dataset.

44. The method of claim 36, further comprising:
identifying a second candidate patient information dataset;
computing a second match score between the non-healthcare dataset associated with the first patient and the second candidate patient information dataset;
responsive to the second match score falling below the threshold, creating or updating the patient information data structure to include an indication that the second candidate patient information dataset is unassociated with the first patient.

45. The method of claim 44, further comprising:
responsive to the second match score falling below the threshold, transmitting to at least one healthcare provider system, at least one of:
a first indication indicative of the second match score falling below the threshold;
a second indication indicative of the first patient information dataset being unassociated with the first patient, or
an augmented patient information dataset unassociated with the first patient.

46. The method of claim 36, further comprising:
identifying a second candidate patient information dataset;
computing a second match score between the second candidate patient information dataset and the first candidate patient information dataset;
responsive to the second match score meeting a second threshold, selecting the second candidate patient information dataset as a second patient information dataset; and
storing the second patient information dataset in the patient information data structure, wherein the patient information data structure associates the second patient information dataset with the first patient.
